(19) United States
(12) Patent Application Publication
(10) Pub. No.: US 2010/0035287 A1
(43) Pub. Date: Feb. 11, 2010

(54) Secreted Luciferase Fluorescent Protein Conjugate Nucleic Acid Construct and Uses Thereof

(75) Inventors: Bakhos A. Tannous, Malden, MA (US); Xandra Breakefield, Newton, MA (US); Jeffrey W. Hewett, Duxbury, MA (US)

Correspondence Address: DAVID S. RESNICK, NIXON PEABODY LLP, 100 SUMMER STREET, BOSTON, MA, 02110-2131, US

(73) Assignee: THE GENERAL HOSPITAL CORPORATION, Boston, MA (US)

(21) Appl. No.: 12/444,068

(22) PCT Filed: Oct. 12, 2007
(86) PCT No.: PCT/US07/21810

§ 371 Date: Apr. 2, 2009

Related U.S. Application Data
(60) Provisional application No. 60/851,110, filed on Oct. 12, 2006.

(51) Int. Cl. C12Q 1/66; C07H 21/00

(52) U.S. Cl. 435/8; 536/23.2

(57) ABSTRACT

The present invention relates generally to methods to monitor the transport of proteins through the secretory pathway, and methods to monitor ER stress. In particular, the present invention relates to methods to monitor, in real-time, the processing of protein through the secretory pathway, which can be monitored both at a subcellular level by florescence visualization and quantitatively by detecting the secreted luciferase reporter protein. The present invention also relates to methods to assess biological processes in cells, in particular the secretory pathway and ER stress, as well as methods to identify agents which augment or inhibit the secretory pathway and/or ER stress. The present invention also relates to compositions and nucleic constructs encoding a secreted luciferase-fluorescent protein conjugate for methods to monitor protein trafficking in the cell by simultaneous detection of fluorescence and luciferase secretion.

### CROSS REFERENCED APPLICATION

This application is a 371 National Phase Entry Application of co-pending International Application PCT/US2007/021810 filed Oct. 12, 2007, which designated the U.S., and claims the benefit under 35 U.S.C 119(e) of U.S. Provisional Patent Application Ser. No. 60/851,110 filed Oct. 12, 2006, the contents of which are incorporated herein by reference in their entirety.

### GOVERNMENT SUPPORT

This invention was made with Government support under NS28384 and 5P01 NS037409 awarded by the National Institutes for Health (NIH) and National Institute of Neurological Disorders and Stroke (NINDS). The Government of the United States has certain rights in the invention.

### FIELD OF THE INVENTION

The invention relates to materials and methods for the identification and assessment of biological systems. More particularly, the invention relates to a system for real-time analysis of biological systems using a secreted luciferase and a fluorescent protein conjugate as a reporter.

### BACKGROUND OF THE INVENTION

The secretory pathway controls processing of a variety of proteins destined for cell membranes and organelles, as well as release from the cell. Proteins typically enter the secretory pathway through binding of a signal sequence at the N terminal region of a protein either co- or post-translationally (Osbourne et al., 2005). Proteins enter the ER through translocons which are closely associated with the ribosomes and ribophorin on the cytoplasmic face and with chaperones, such as calreticulin and PDI on the lumenal face. Following post-translational modification, correct folding and multimerization, proteins leave the ER within vesicles through exit sites which move onto fuse with the Golgi apparatus. Misfolded or mutant proteins can be shuttled back out the translocon for degradation by proteosomes in the cytoplasm (Tsai et al., 2002). Vesicles leaving the Golgi can take proteins to other vesicular or organelle compartments or mediate release into the extracellular space via fusion of vesicles with the plasma membrane.

Two assays are commonly used to monitor transit of proteins through the secretory pathway in mammalian cells using reporter proteins which are delivered to cells in culture by transduction of expression cassettes, such as measurement of secreted alkaline phosphatase (Yang et al., 1997) and use of a temperature-sensitive viral glycoprotein-GFP fusion protein (Presley et al., 1997), discussed in more detail below.

In one, a temperature sensitive form of the vesicular somatitis virus (VSV) glycoprotein is fused to GFP, such that at high temperature (40 C) is accumulates as a misfolded complex in the ER and when the temperature is reduced (32 C or less) it moves onto the Golgi and then out to the plasma membrane (Presley et al., 1997). There are several advantages to this system in that the movement of the protein can be synchronized by temperature shifts and visualized by fluorescent microscopy, with the disadvantage that the efficiency of secretion can not be quantitated.

In the other, the normally secreted enzyme, alkaline phosphatase (SEAP, Clontech, CA) is expressed in cells and activity in the medium is measured using either a chemiluminescent or fluorescent substrate. In this case there is no visualization of movement through the cells, but levels of secretion can be quantitated with high sensitivity (Yang et al., 1997). Interference by endogenous alkaline phosphatase can be minimized by the relative stability of SEAP to heat and its unique resistance to L-homoarginine.

An ideal system to monitor the processes of proteins through the protein secretion pathway would encompass both qualitative assessment of the location of the secreted protein by visualization and quantification of the level of protein secretion. However, to date such a system that efficiently accomplishes the goals of dual quantification with visualization of protein secretion in real time has not been described.

### SUMMARY OF THE INVENTION

The present invention is based, on part, on the discovery that a secreted form of luciferase conjugated to a fluorescent protein is capable of acting as a dual reporter for the assessment of biological processes in cells. The analysis of the secreted form of the luciferase-fluorescent protein conjugate can be monitored without the need to lyse the cells. This permits the convenient detection, using conventional calorimetric methods and/or fluorescent assays, of the luciferase-fluorescent protein conjugate produced by the cell at different time points. Therefore, one embodiment is a method for real-time monitoring of biological systems using the luciferase-fluorescent protein conjugate protein.

In certain embodiments, repeated monitoring and quantization can be easily performed be by taking aliquots of the cell culture media over time. In other embodiments, since the cells are not disrupted during assay, they can be used for other assays in parallel. Since the luciferase used in the invention is naturally secreted, the methods can be performed on small samples of the conditioned media, without the need to lyse the cells, makes the methods much faster and more convenient than other assays with other luciferases such as firefly which is used in the SUPERLIGHT™ luciferase reporter gene assay (Bioassays, CA) where cell lysis is required.

In one embodiment of the invention, the luciferase-fluorescent protein conjugate is used as dual reporter for analysis of biological systems in cells, where qualitative analysis of the cells can be performed by assessment of the sub-cellular and cellular localization of the luciferase-fluorescent protein conjugate and quantitative analysis of the cells can be performed by bioluminescence of the luciferase-fluorescent protein conjugate. Another embodiment is the dynamic monitoring of the expression and/or localization of the luciferase-fluorescent protein conjugate within the cell by fluorescence, and the monitoring of the expression and/or secretion of the luciferase-fluorescent protein conjugate by bioluminescence.

In one embodiment the luciferase-fluorescent protein conjugate is expressed within cells by a nucleic acid construct encoding the luciferase-fluorescent protein conjugate operatively linked to a regulatory sequence. In one embodiment, the luciferase is a secreted form of luciferase. Any secreted form of luciferase is useful in the methods as disclosed herein, and includes fragments,variants and recombinant forms of luciferase proteins. In some embodiments, the secreted luciferase is Gaussia luciferase. In another embodiment, the Gaussia luciferase is humanized Gaussia luciferase for expression in mammalian cells. Without being limited to theory, Gaussia luciferase is 2000-fold more sensitive than firefly luciferase (FLuc) and 200-fold than sensitive than alkaline phosphatase (SEAP) in medium. Further, Gaussia luciferase is linear over at-least 10⁷ fold range, whereas SEAP is only linear over a 10⁴ fold range.

In another embodiment the nucleic acid construct is introduced into cells by means of a vector. In another embodiment, the vector is a viral vector.

In anther embodiment, the sub-cellular and cellular localization and bioluminescence of the luciferase-fluorescent protein conjugate can be assessed in the response to one or more environmental stimuli. In such an embodiment, a change in any; fluorescence; pattern of fluorescence; and/or bioluminescence of the Gaussia luciferase-fluorescent protein conjugate from the cell indicates an effect of an environmental stimuli on the biological processing of the cell.

In one embodiment, the methods of the invention can be used for monitoring the protein secretion or protein secretory pathway. In such an embodiment, the methods of the invention described herein are advantageous over previous secretory pathway assays, in that the methods of the present invention enable real time analysis of the secretory pathway by combining the visualization of the Gaussia luciferase-fluorescent protein conjugate with quantitation of the level of secretion of Gaussia luciferase-fluorescent protein conjugate. The methods in such an embodiment enable monitoring of the primarily entry of the secreted protein (in this case Gaussia luciferase-fluorescent protein conjugate) into the endoplasmic reticulum (ER) and co-localization with ER, as assessed by a punctuate localization pattern which co-staining with ER markers. Thus, the methods of the invention have the advantage of analyzing the processing of a secreted protein (in this case Gaussia luciferase-fluorescent protein conjugate) at distinct positions in the secretory pathway, namely the ER entry points, combined with the ability to monitor quantitatively the level of secretion by bioluminescence. Thus, the methods of this invention are the most sensitive assay to analyze the secretory pathway in living cells.

In another embodiment, the methods of the invention can be used in the assessment of promoter activities and/or regulatory sequences. In such an embodiment, a Gaussia luciferase fluorescent protein conjugate is operatively linked to a regulatory sequence and the activity of regulatory sequence is determined by fluorescence and bioluminescence of the Gaussia luciferase-fluorescent protein conjugate. In a related embodiment, the effects of one or more environmental stimuli on the activity of the regulatory sequence assessed by changes in fluorescence and/or bioluminescence in response to at least one environmental stimuli.

In another embodiment, the methods of the invention can be used to study transcriptional activation in cells. In such an embodiment, the regulatory sequences operatively linked to the Gauissa luciferase fluorescent conjugate protein are responsive to particular transcription factors and/or regulatory proteins. In such an embodiment, the transcriptional activity is determined by fluorescence and/or bioluminescence of the Gaussia luciferase-fluorescent protein conjugate. In a related embodiment, the effect of environmental stimuli on the transcriptional activity is assessed by changes in fluorescence and/or bioluminescence in response to at least one environmental stimuli.

In another embodiment, the methods of the invention can be used to study signaling pathways in cells. In such an embodiment, the regulatory sequences operatively linked to the Gauissa luciferase fluorescent conjugate protein are responsive to particular signaling molecules. In such an embodiment, the activity of the signaling pathway can be determined by fluorescence and/or bioluminescence of the Gaussia luciferase-fluorescent protein conjugate. In a related embodiment, the effect of environmental stimuli on the activity of the signaling pathway is assessed by changes in fluorescence and/or bioluminescence in response to the environmental stimuli.

In another embodiment, the methods of this invention can be used to monitor cell viability. In such an embodiment, as the secretion of Gaussia luciferase-fluorescent conjugate protein is proportion to the cell number and the fluorescence and/or the bioluminescence can be used as a direct measure the number of cells. In a related embodiment, the effect of environmental stimuli on cell viability can be assessed by changes in fluorescence and/or bioluminescence in response to the environmental stimuli. Increases in fluorescence and/or bioluminescence indicate increased cell proliferation and/or altered cell viability or cell survival, whereas decreases in fluorescence and/or bioluminescence indicate cell death and/or decreased proliferation.

In another embodiment, the methods can be used to study protein-protein interactions in cells by use of BRET (bioluminescence-Energy Transfer system) (see Boute et al (2002) Trends Pharmacol. Sci. 32;351-354). In a related embodiment, the effect of environmental stimuli on protein-protein interactions is assessed by changes in BRET signals, fluorescence and/or bioluminescence in response to the environmental stimuli.

One aspect of the present invention related to a nucleic acid construct comprising; a nucleic acid sequence encoding a secreted luciferase-fluorescent protein conjugate; a nucleic acid sequence encoding at least one regulatory sequence, wherein the nucleic acid encoding a secreted luciferase-fluorescent protein conjugate is operatively linked to at least one of regulatory sequence. In some embodiments, the secreted luciferase-fluorescent protein conjugate is a Gaussia luciferase-fluorescent protein conjugate, or a fragment or variant thereof. In some embodiments, the nucleic acid sequence encodes at least one multiple cloning site for the introduction of target nucleic acid sequences, and at least one at least one target nucleic acid sequence. In some embodiments, the construct further comprising a nucleic acid sequence encoding at least one internal ribosome entry site (IRES) and/or at least one nucleic acid sequence encoding a marker gene for identification of cells containing the nucleic acid construct. In some embodiments, such a marker gene is a nucleic acid sequence encodes an additional fluorescent protein, wherein the fluorescent protein is spectrally distinguishable from the fluorescent protein in the Gaussia luciferase-fluorescent fusion protein.

In some embodiments, a Gaussia luciferase-fluorescent protein conjugate comprises Gaussia luciferase (GLuc) and has the nucleic acid sequence as set forth in SEQ ID NO:1.In some embodiments, the nucleic acid sequence for Gaussia luciferase (GLuc) is codon optimized for mammalian gene expression, for example for expression in human cells, for example the Gaussia luciferase is humanized is humanized Gaussia luciferase (hGLuc) and has the nucleic acid sequence as set forth in SEQ ID NO.3.

In some embodiments, the Gaussia luciferase-fluorescent protein conjugate is a fusion protein, and in alternative embodiments, the Gaussia luciferase-fluorescent protein conjugate is conjugated by chemical means.

In some embodiments, the nucleic acid construct is present or is a vector, for example an expression vector. In alternative embodiments, the vector is a viral vector, for example, but not limited to viral vector such as, a lentivirus vector, retroviral vector, lentivirus vector, herpes simplex viral vector, adenovirus vector, adeno-associated virus (AAV) vectors, EPV, EBV or variants or derivatives thereof.

Another aspect of the present invention relates to a method of analyzing a cell comprising the steps of: (i) introducing the nucleic acid construct as disclosed herein comprising a nucleic acid sequence encoding a secreted luciferase-fluorescent protein conjugate; a nucleic acid sequence encoding at least one regulatory sequence, wherein the nucleic acid encoding a secreted luciferase-fluorescent protein conjugate is operatively linked to at least one of regulatory sequence into a cell; (ii) expressing the secreted luciferase-fluorescent protein conjugate; (iii) measuring a fluorescence signal from the secreted luciferase-fluorescent protein conjugate; and/or measuring the bioluminescence signal from the secreted luciferase-fluorescent protein conjugate.

Another aspect of the present invention relates to a method of measuring ER-stress in a cell comprising the steps of; (i) introducing the nucleic acid construct as disclosed herein comprising a nucleic acid sequence encoding a secreted luciferase-fluorescent protein conjugate into a cell; (ii) expressing the secreted luciferase-fluorescent protein conjugate; (iii) measuring the level of fluorescence signal from the secreted luciferase-fluorescent protein conjugate; and/or (iv) measuring the level of bioluminescence signal from the secreted luciferase-fluorescent protein conjugate, wherein the level of fluorescence signal from the secreted luciferase-fluorescent protein conjugate and/or level of bioluminescence signal from the secreted luciferase-fluorescent protein conjugate is a measure of ER stress in the cell. For example, but not limited to, such a method is useful to determine if a cell is undergoing ER stress, as disclosed in the examples, where presence of ER-stress is detected by a reduction of luciferase signal as compared to the absence of ER stress. In such embodiments, the secreted luciferase-fluorescent protein conjugate is a Gaussia luciferase-fluorescent protein conjugate or any other secreted luciferase protein or fragment or variant thereof conjugated to a fluorescent protein, or fragment thereof.

In some embodiments, measuring a fluorescence signal comprises the step of illuminating the cell at an excitation wavelength sufficient for excitation of the fluorescent protein encoded by the nucleic acid of the fluorescent protein conjugated to the nucleic acid encoding the secreted luciferase. In some embodiments, the method further comprises the step of identifying an intracellular location of the protein expressed by nucleic acid sequence encoding secreted luciferase-fluorescent protein conjugate. In some embodiments, the light emitted by the fluorescent protein is detected by methods comprising; fluorimetry; FACs; microscopy techniques and other techniques commonly known by persons of ordinary skill in the art. In some embodiments, the methods as disclosed herein further comprise contacting the cell with a bioluminescence substrate and measuring the bioluminescence. In some embodiments the methods as disclosed herein further comprise harvesting the supernatant from the cells, and contacting the supernatant with bioluminescence substrate and measuring the bioluminescence.

In some embodiments, methods to measure bioluminescence are commonly known by persons of ordinary skill in the art and include, for example but not limited to measuring the bioluminescence by detecting luciferase using a microplate luminometer or using a CCD imaging system. In some embodiments, the methods as disclosed herein further comprise measuring an additional signal from the cell, wherein the additional signal measures the signal from the marker gene to identify the cells containing the vector.

In some embodiments, the methods as disclosed herein for analyzing a cell or ER-stress in a cell comprises a nucleic acid construct comprises a regulatory sequence operatively connected to a coding portion encoding the secreted luciferase-fluorescent protein conjugate, such as Gaussia luciferase-fluorescent protein conjugate; and the method further comprising the step of exposing the regulatory sequence to at least one environmental stimulus, whereby fluorescent signal and/or luciferase signal from the secreted luciferase-fluorescent protein conjugate measures an effect of the environmental stimulus on the activity of the regulatory sequence.

In some embodiments, a regulatory sequence is selected from a group comprising; promoters; enhancers; 5′ untranslated regions (5′ UTR); 3′ untranslated regions (3′UTRs); and repressor sequences, or in some instances, a regulatory sequence is for example but not limited to a promoter, or constitutively active promoter In some instances, the regulatory sequences are regulated by the stimulus, such as environmental stimuli, and in other embodiments, the regulatory sequences are not regulated by the stimulus.

In some embodiments, an environmental stimulus comprises contacting the cell with at least one compound of interest. In some embodiments of the methods as disclosed herein, the environmental stimulus comprises contacting the cell with at least one protein and/or at least one nucleic acid sequence, for example, such nucleic acid sequence includes, but is not limited to nucleic acid sequences encoding proteins or fragments thereof, small inhibitory nucleic acid molecules, RNAi, shRNA, siRNA, antisense nucleotides, PNA, DNA, pcPNA, miRNA, antigomers, or analogues and variants thereof.

In some embodiments, the environmental stimuli is a protein is a protein and/or peptide of interest, or fragment thereof, for example a protein and/or peptide of interest, or fragment thereof, can be, for example but are not limited to mutated proteins, a protein fused to one or more other proteins, mutated proteins fused to one or more other proteins, antibodies, and fragments and variants thereof.

In some embodiments, an environmental stimuli is encoded by a target nucleic acid sequence in the nucleic acid construct as disclosed herein. Alternatively, the environmental stimulus can be encoded in a separate nucleic acid construct.

In some embodiments, the methods to analyze cells and ER-stress in a cell can optionally further comprising the steps of (i) introducing a nucleic acid encoding the environmental stimulus into a cell; and (ii) expressing the environmental stimulus in the cell.

In further embodiments, the fluorescence and bioluminescence of the secreted luciferase-fluorescent protein conjugate or Gaussia luciferase-fluorescent protein conjugate is substantially unaffected by the environmental stimuli.

Other aspects of the present invention provide use of the nucleic acid construct as disclosed herein to study the effects of modification of a regulatory sequence on the activity of a regulatory sequence in the cell, and for example, to the study the effects of environmental stimuli on a regulatory sequence and/or promoter activity in the cell.

In further embodiments, the methods of the present invention as disclosed herein are useful in the study the effects of an intrinsic environmental stimuli on secretory pathways in the cell, wherein any change in the secretory pathway is detected by the fluorescent signal and/or cellular localization of the secreted luciferase-fluorescent protein conjugate or Gaussia luciferase-fluorescent protein conjugate and/or any change in the luciferase signal. Alternatively, the methods of the present invention as disclosed herein are useful in the study the effects of environmental stimuli on secretory pathways in the cell, wherein any change in the secretory pathway is detected by the fluorescent signal and/or cellular localization of the Gaussia luciferase-fluorescent protein conjugate and/or any change in the luciferase signal.

In further embodiments, the methods of the present invention as disclosed herein are useful in the study of numerous biological process, for example but not limited to; the study the effects of environmental stimuli transcriptional activity in the cell; the study the effects of environmental stimuli on signaling pathways in the cell; the study the effects of environmental stimuli on cell viability; and the study of environmental stimuli on ER stress.

In further embodiments, the detection of reporter segment signal is carried out with cells placed on the surface of a microscope slide, or with cells plated in a well of a microtitre plate, or any other method commonly known by persons of ordinary skill in the art.

Another aspect of the present invention relates to a vector comprising the nucleic acid construct as disclosed herein, such as the nucleic acid construct comprising; a nucleic acid sequence encoding a secreted luciferase-fluorescent protein conjugate; a nucleic acid sequence encoding at least one regulatory sequence, wherein the nucleic acid encoding a secreted luciferase-fluorescent protein conjugate is operatively linked to at least one of regulatory sequence. Another aspect of the present invention relates to a cell comprising such a vector, for example a mammalian cell. In some embodiments, the cell can be in vivo, in vitro or ex vivo. In some embodiments, the cell can be a human cell.

### BRIEF DESCRIPTION OF FIGURES

FIG. 1 shows the validation of Gluc secretion assay—linearity with cell number and time. Control (HF24, circle) and DYT1 ( FFF13111983, square) fibroblasts were infected with a lentivirus vector encoding Gluc-IRES-cerulean to achieve infection of >95%. Panel 1A shows infected cells were plated 24 hrs after infection at different densities per well and 24 hrs after plating luciferase activity in the medium was quantitated. Panel 1 B shows infected cells were plated at a density of 2.5×10³ cells per well and luciferase activity in the media was quantitated 24, 48 and 72 hrs after plating.

FIG. 2 shows DYT1 fibroblasts have a lower rate of secretion of Gluc as compared to control fibroblasts. Cells from 3 DYT1 and 3 control lines were infected with the lentivirus vector encoding Gluc-IRES-cerulean and replated 24 hrs after infection at 2×10⁵ cells per well. Panel 2A shows twenty four hrs after plating luciferase activity was determined in the medium. The experiment was repeated 2 times in triplicate for each cell line and the mean is shown ±S.D. The average of the means of the DYT1 lines and the control lines was significantly different at p<.004. Panels 2B and 2C show examples of the high infectivity of control (HF6) and DYT1 (HF48) lines as assessed by cerulean fluorescence. Magnification 100×.

FIG. 3 shows levels and distribution of Gluc-YFP in control and DYT1 media and cells. Cells were infected with lentivirus vector encoding Gluc-YFP (SEQ ID NO.5) and 24 hrs later cells were plated at 2×10³/well. Panel 3A shows 24 hrs after plating luciferase activity was assessed in the media (top) and in living cells (bottom). In both control (HF6) and DYT1 (HF47) cells 5% activity was retained in the cells with 95% being secreted. Panel 3B shows although there was 2-3 times as much luciferase activity in control cells as compared to DYT1 cells, western blot analysis indicated similar levels of Gluc-YFP protein in both cell types using alpha-tubulin as a index of loading. Panel 3C shows immunocytochemistry of the Gluc-YFP fusion protein revealed a punctuate reticular pattern in control cells (top), while DYT1 cells showed a more diffuse, apparently cytoplasmic staining. Magnification 100×. Note. This was repeated in 3 DYT1 and 2 control lines with similar results.

FIG. 4 shows co-staining of Gluc-YFP puncta in control cells with ER markers. Control cells ((HF24) were infected with Gluc-YFP lentivirus vector and 24 hrs later dual immunocytochemistry was carried out for GFP (4A, 4D, 4G) and other ER markers (4B, 4E, 4H) with merged images shown in the right most panels. Panels 4A -4C shows Gluc-YFP puncta were seen to be beaded along the ER proper as visualized with staining for PDI. 4D -4F) Many Gluc-YFP puncta appeared to co-localize with the translocon marker, Sec61α. Panels 4G-4H show some of the Gluc-YFP puncta also appeared to co-localize with the ER-to-Golgi marker, COPII. Magnification 100×.

FIG. 5 shows differential solubilization of control and DYT1 fibroblasts expressing Gluc-YFP reveals differential distribution. Monolayers of control (HF24) and DYT1 (FFF076111984) cells infected with Gluc-YFP-IRES-cerulean lentivirus vector were sequentially solubulized 24 hrs post-infection with digitonin (cytoplasmic fraction), Triton X-100 (ER fraction) and SDS (remaining proteins) and extracts resolved by SDS-PAGE with western blotting for the cytoplasmic marker—GAPDH, ER markers—calnexin and torsinA, the reporter protein—Gluc-YFP. Note. This was repeated in 2 additional DYT1 and control lines with similar results.

FIG. 6 shows immune precipitation with antibodies to torsinA and western blot analysis with antibodies to GFP. Control (HF19) and DYT1 (HF47) cells were infected with Gluc-YFP-IRES-cerulean or YFP-IRES-cerulean (control) lentivirus vector and 72 hrs later lysates were prepared and immune precipitated for torsinA. Lysates and pellets were resolved by SDS-PAGE and western blotting was carried out with antibodies to torsinA and GFP.

FIG. 7 shows monitoring of promoter activity with Gluc. Gli36 human glioma cells were infected with HSV amplicon vector carrying hGluc cDNA under the control of CMV promoter. Promoter activity was monitored overtime by taking an aliquot of the conditioned media, adding coelenterazine and measuring photon counts for 10 sec using a luminometer.

FIG. 8 shows use of Gluc for monitoring transcriptional activation. Different promoters responsive to transcription factors were cloned upstream of hGluc cDNA and there activity was measured 24 hrs after infection with each construct by taking an aliquot of the conditioned media, adding coelenterazine and measuring photon counts for 10 sec. RLU=relative light unit.

FIG. 9 shows Monitoring of NF-kB signaling with hGluc. 293T cells were transfected with a plasmid carrying hGluc under the control of 5NFkB responsive elements. FIG. 9A shows 24 hrs later, cells were treated with different concentrations of bleomycin (9A) or 1 uM of etoposide or doxorubicin (9B) or irradiated at different doses (9B and 9C) and the Gluc activity in the conditioned medium was measured either 24 hrs later (9A and 9B) or at different time points (9C). Signals are normalized to basal level of hGluc activity without treatment.

FIG. 10 shows monitoring cell viability with Gluc. Gli36 human glioma cells stably expressing Gaussia luciferase were plated in 96 well plate and infected with an HSV amplicon carrying either firefly luciferase (dash line) or a mutant form of human brain sodium channel (BNac1, solid line) which is constitutively open and kills cells (solid line, Tannous and Breakefield, in preparation) under the control of CMV promoter. Cell viability was monitored overtime by taking an aliquot of supernatant at different time points from the same well, adding coelenterazine and measuring photon counts for 10 sec using a luminometer.

FIG. 11 shows processing and secretion of Gluc via the secretory pathway. Bioluminescence was monitored from media of Gluc expressing cells in the presence or absence (untreated) of agents which inhibit the secretory pathway. Nocodazone disrupts microtubule function and BrefeldinA disrupts golgi function.

FIG. 12 shows Gluc expression has no effect on the unfolded protein response (UPR). Immunobloting for GRP76 of protein lysates from cells not expressing Gluc in the absence (untreated) or presence of tunicamycin, an agent which upregulates the UPR, compared with untreated cells expressing the Gluc-YFP conjugate protein.

FIG. 13 shows sensitivity of Gluc versus SEAP. Signal intensity of 293T human fibroblast cells 24 hrs after transfection with either Gluc or SEAP expressing constructs. Signal intensity for either Gluc or SEAP activity in media collected from the cells 24 hrs after transfection was compared for Gluc and SEAP transfected cells respectively.

FIG. 14 shows Gluc as a reporter in mammalian cells. Panel 14A shows a schematic representation of the expression cassettes for Gluc-IRES-CFP and Panel 14B shows Gluc-YFP fusion cloned in the CSCW lentivirus vector. Panel 14C shows high infection rate of cells with lentivirus vectors (M.O.I.=30) as monitored by cerulean fluorescence. Scale bar, 100 μm. Panel 14D shows Levels of Gluc activity in cells vs. medium vs. cells+medium. 293T cells were infected with the lentivirus vector carrying the expression cassette for Gluc-IRES-CFP and 20,000 cells were plated in wells of a 96-well plate. 48 h post-infection, new medium was added to the wells and Gluc activity was measured 24 hrs later in conditioned medium, viable washed cells or cells+conditioned medium after adding 2.5 μM coelenterazine.

FIG. 15 shows linearity and sensitivity of the Gluc assay. Panel 15A shows 293T cells were infected with lentivirus vector carrying the expression cassette for Gluc-IRES-CFP and 20,000 cells were plated in wells of 96-well plate. Forty-eight hrs later, Gluc activity was monitored overtime in 10 μL of conditioned medium after addition of 20 μM coelenterazine. The release of Gluc to the conditioned medium is linear with time. Panel 15B shows 293T cells were co-transfected with pHGC-Fluc and either pHGC-Gluc or pSEAP expression plasmids. Gluc and SEAP activities were assayed in conditioned medium 24 hrs later. The signal over background ratio (S/B) values, normalized to the intracellular levels of Fluc, are plotted against the number of cells. The Gluc assay can detect a single cell with S/B of 40 whereas the SEAP assay requires 20,000 cells to get similar S/B under similar assay conditions. The mean±S.E.M. is presented on the graphs (n=3).

FIG. 16 shows Gluc as a reporter to monitor secretory pathway. 293T cells were infected with the lentivirus vector expressing Gluc and were plated in wells of 96-well plate. Cells were treated for 24 h with different drugs which interfere with the secretory pathway. Panel 16A shows cell-free conditioned media were assayed for Gluc activity which showed that Gluc secretion is decreased upon blocking the secretory pathway. *p<0.05 as predicted by student T-test. Panel 16B shows immunocytochemistry on cells expressing Gluc with and without treatment with BFA showing that Gluc (cy3, red) co-localizes with the ER marker PDI (Alexa488, green). Scale bar, 10 μm.

FIG. 17 shows Gluc-YFP fusion to visualize secretory pathway in real-time. Panel 17A shows uninfected 293T cells or cells infected with a lentivirus vector expressing Gluc-YFP fusion were lysed and analyzed by western blotting with anti-Gluc antibody. Panel 27B shows cells expressing Gluc-YFP fusion were treated with BFA or nocodazole and their conditioned medium were assayed for Gluc activity 24 h later. *p<0.01 as predicted by student T-test. Panel 17C shows Fluorescence microscopy of a single live cell expressing Gluc-YFP and either untreated or treated with BFA showing that this fusion is trapped in the ER upon BFA treatment. Scale bar, 10 μm.

FIG. 18 shows monitoring of ER stress with Gluc. Panels 18A to 18C shows 293T cells expressing Gluc were subjected to no or different concentrations of DTT to induce ER stress. Panel 18A shows real-time RT-PCR for spliced XBP-1 mRNA which increased in response to >1 mM DTT 4 h after treatment. Fold induction is calculated with respect to the control non-infected/non-treated cells. Panel 18B shows Western blot analysis showing upregulation of BiP levels in response to ER stress 24 h after treatment at >1 mM DTT. Blot is also probed with Gluc antibody as well as alpha-tubulin antibody for equal loading. Panel 18C shows conditioned medium assayed for Gluc activity 4 h after DTT treatment showing that Gluc secretion is decreased in response to ER stress (panel 18D). Untreated or treated cells with 1 mM DTT were monitored overtime for the level of Gluc secretion by assaying an aliquot of the conditioned medium for Gluc bioluminescence. The mean±S.E.M. is presented on the graphs (n=3), with *p<0.01 as compared to the non-treated cells and as calculated by the student's t-Test. NS=not significant

The nucleic acid sequence set forth in SEQ ID NO.1 is the nucleic acid sequence of the gene encoding Gaussia luciferase (GLuc), deposited under GenBank database accession number AY015993.

The amino acid sequence set for the in SEQ ID NO.2 is the amino acid sequence of Gaussia luciferase (GLuc) protein, deposited under GenBank database accession number AAG54095.

The nucleic acid sequence set forth in SEQ ID NO.3 is the nucleic acid sequence of the gene encoding humanized Gaussia luciferase (hGLuc), which has been human codon optimized.

The amino acid sequence set forth in SEQ ID NO.4 is the amino acid sequence of humanized Gaussia luciferase (hGLuc) protein, which has been human codon optimized.

The nucleic acid sequence set forth in SEQ ID NO.5 is the nucleic acid sequence of the gene encoding Gaussia luciferase (GLuc)-YFP fusion protein.

### DETAILED DESCRIPTION OF THE INVENTION

The present invention includes a number of different assays. In one embodiment, the assay can be used to monitor the transit of proteins through the secretory pathway. This is of a special interest for high throughput screening of different drugs affecting the secretion of proteins. In another embodiment, this assay is used for monitoring of promoter and/or regulatory sequence activities and their response to different stimuli such as drug or ionizing radiation in mammalian cells. In another embodiment, the invention can be used to monitor transcriptional activation and/or signaling pathways in the presence and absence of environmental stimuli. In another embodiment, the assay is used to monitor cell viability and cell death, in the presence and absence of environmental stimuli. In another embodiment, the assay can be used to monitor protein-protein interactions.

The nucleic acid construct of the present invention comprises nucleic acid, typically DNA, with, at its 5′ end in the direction of transcription, the regulatory sequence which allows for induced transcription of the nucleic acid encoding a secreted form of a Luciferase protein. In one embodiment, the luciferase is Gaussia luciferase. In another embodiment, the Gaussia luciferase is fused, in-frame, to an appropriate fluorescent protein. In alternative embodiments, the Gaussia luciferase is conjugated to a fluorescent protein by any means known to persons skilled in the art. Also, alternative embodiments include any secreted luciferase known to those skilled in the art. Generally speaking, the nucleic acid constructs are expressed in the cells to be tested by means of an expression vector. Typically, but not exclusively the cells are of mammalian origin and the expression vectors chosen is one which is suitable for expression in the particular cell type.

The nucleic acid construct of the present invention can be introduced as one or more DNA molecules or constructs. The construct can be prepared in conventional ways, where genes and modulatory elements may be isolated, as appropriate, ligated, cloned in appropriate cloning host, analyzed by restriction or sequencing or other conventional means known to those skilled in the art. Particularly, using PCR, individual fragments including all or portions of the functional regulatory elements may be isolated where one or more mutations may be introduced using methods described below. The construct(s) once completed and demonstrated to have the appropriate sequences may then be introduced into the host cell by any conventional means.

The term “luciferase” or “luciferases” used interchangeably herein, refers to an enzyme or photoprotein that catalyzes a bioluminescent reaction [a reaction that produces bioluminescence]. Luciferases are catalytically and are unchanged during bioluminescence. Luciferase photoproteins to which luciferin is non-covalently bound, are changed, such as by the release of luciferin during the bioluminescence reaction. “Luciferin” used to herein, refers any compound that, in the presence of any necessary activators, catalyze the oxidation of a bioluminescence substrate in the presence of molecular oxygen, whether free or bound, from a lower energy state to a higher energy state, such that the substrate upon return to the lower energy state, emits light.

“Gauissa Luciferase” used interchangeably as “Gluc” or “GLuc” herein, refers to the luciferase enzyme isolated from member of the genus Gaussia or an equivalent molecule obtained from any other source, such as from another related copepod, or has been prepared synthetically. It is intended to encompass Gauissa luciferase with conservative amino acid substitutions that do not substantially alter activity. Suitable conservative substitutions of amino acids are known to those skilled in the art and may be made generally without altering the biological activity of the resulting molecule. Those of skill in the art, recognize that, in general, single amino acid substitutions in non-essential regions of the polypeptide do not alter biological activity (see e.g. Watson et al, Molecular biology of the gene, 4^{th} Ed, 1987, p 224)

The DNA encoding Gauissa Luciferase is available commercially, for example ProLume Ltd and Nanotlight. Alternatively, methods of isolating the DNA encoding Gaussia luciferase from natural sources are described in U.S. Pat. No. 6,436,682 and are incorporated herein for reference. Methods of producing mutants containing a predetermined nucleotide sequence are well known in the art. Two widely known methods are Kunkel mutagenesis and PCR mutagenesis. A detailed description can be found in Current Protocols in Molecular Biology, Wiley Interscience, 1987, sections 8.1 and 8.5 respectively. Kunkel mutagenesis is also described in an article by Kunkel in Proc. Acad. Natl. Sci, USA, 82;488-492, while PCR is discussed in an article by Saiki et al, Science 239,487-491.

The term “humanized Gaussia luciferase” refers to a humanized form of a Gauissa Luciferase nucleic acid sequence, in which the nucleic acid sequence has been modified for expression in mammalian cells. A detailed description for the methods for producing humanized Gaussia luciferase can be found in Tannous et al, (2005) Mol Therapy, 11;435-443.

The term “fluorescent protein” refers to a protein that possesses the ability to fluorescence (i.e., to absorb energy at one wavelength and emit it at another wavelength). For example, a green florescent protein refers to a polypeptide that has a peak in the emission spectrum at about 510 nm. Any fluorescent protein known by persons of ordinary skill in the art are encompassed for use in the methods, nucleic acid construct and compositions as disclosed herein. The fluorescence proteins can be used as a fluorescent label or marker and in any application in which such labels would be used, such as immunoassays, CRET, FRET, and FET assays, and in the assays such as the BRET (Bioluminescence Resonance Energy Transfer) in U.S. Pat. No. 6,436,682 which is incorporated herein by reference, refers to any method in which luciferase is used to generate light upon reaction with luciferin which is non-radioactively transferred to a fluorescent protein.

The term “conjugate” refers to the attachment of two or more proteins joined together to form one entity. The proteins may attached together by linkers, chemical modification, peptide linkers, chemical linkers, covalent or non-covalent bonds, or protein fusion or by any means known to one skilled in the art. The joining may be permanent or reversible. In some embodiments, several linkers may be included in order to take advantage of desired properties of each linker and each protein in the conjugate. Flexible linkers and linkers that increase the solubility of the conjugates are contemplated for use alone or with other linkers are incorporated herein. Peptide linkers may be linked by expressing DNA encoding the linker to one or more proteins in the conjugate. Linkers may be acid cleavable, photocleavable and heat sensitive linkers.

The term “fusion protein” refers to a recombinant protein of two or more proteins. Fusion proteins can be produced, for example, by a nucleic acid sequence encoding one protein is joined to the nucleic acid encoding another protein such that they constitute a single open-reading frame that can be translated in the cells into a single polypeptide harboring all the intended proteins. The order of arrangement of the proteins can vary. As a non-limiting example, the nucleic acid sequence encoding the Gaussia Luciferase-fluorescent fusion protein is derived from the nucleotide sequence of Gaussia Luciferase fused in frame to an end, either the 5′ or the 3′ end, of a gene encoding a fluorescent protein. In this manner, on expression of the gene, the reporter protein is functionally expressed and fused to the N-terminal or C-terminal end of the Gaussia luciferase. Modification of the fluorescent protein is such that the functionality of the fluorescent protein remains substantially unaffected by fusion of the Gaussia Luciferase protein to the fluorescent protein. In one embodiment, the humanized Gaussia Luciferase is fused in-frame at the carboxyl terminal with enhanced YFP.

The term “linker” refers to any means to join two or more proteins by means other than the production of a fusion protein. A linker can be a covalent linker or a non-covalent linker. Examples of covalent linkers include covalent bonds or a linker moiety covalently attached to one or more of the proteins to be linked. The linker can also be a non-covalent bond, e.g. an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, Gaussia luciferase and/or the fluorescent protein can be modified by oxidation, hydroxylation, substitution, reduction etc. to provide a site for coupling. It will be appreciated that modification which do not significantly decrease the function or Gaussia luciferase and fluorescent protein are preferred.

The term “regulatory sequences” is used interchangeably with “regulatory elements” herein refers element to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequence to which they are operatively linked. Regulatory sequence often comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, an optional operate sequence to control transcription, a sequence encoding suitable mRNA ribosomal binding sites, and sequences to control the termination of transcription and/or translation. Regulatory sequences are selected for the assay to control the expression of Gaussia Luciferase-fluorescent fusion protein in a cell-type in which expression is intended.

Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc.

As used herein, a “promoter” or “promoter region” or “promoter element” used interchangeably herein, refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be cis-acting or may be responsive to trans-acting factors. Promoters, depending upon the nature of the regulation may be constitutive or regulated.

The term “constitutively active promoter” refers to a promoter of a gene which is expressed at all times within a given cell. Exemplary promoters for use in mammalian cells include cytomegalovirus (CMV), and for use in prokaryotic cells include the bacteriophage T7 and T3 promoters, and the like.

The term “operatively linked” or “operatively associated” are used interchangeably herein, and refer to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined.

The term “target sequence” refers to any nucleic acid sequence that functions as environmental stimuli. Nucleic acid sequences functioning as environmental stimuli include, but are not limited, to sequences encoding a protein or peptide of interest or fragment thereof, or a nucleic acid sequence that function directly as environmental stimuli. The target nucleic acid sequence can be DNA and/or RNA of combinations thereof. Nucleic acid sequences that function directly as environmental stimuli include sequences that inhibit transcription and/or translation, for example but not limited to nucleic acid inhibitors such as; antisense oligonucleotides; RNAi; shRNAi; siRNA; microsatelite RNAi (mRNAi); etc.

The term “IRES” refers to internal ribosome entry sites (see Kozak (1991) J. Biol. Chem. 266′19867-70) are sequences encoding consensus ribosome binding sites, and can be inserted immediately 5′ of the start codon and/or regulatory sequences or elsewhere 5′ of nucleic acid sequences encoding genes or marker genes to enhance expression of the downstream nucleic acid sequence. The desirably of, or need for, such modification may be empirically determined.

The term “marker gene” refers to any gene whose expression can be detected. Many such genes are known in the art. One or more marker genes allow for the selection of host cells which contain the nucleic acid construct. Cells can be screened for a marker present in the construct. Various markers include hprt, neomycin resistance, thymidine kinase, hygromycin resistance etc, and various cell-surface markers such as Tac, CD8, CD3, thy1, NGF receptor etc.

The term “cell” used herein refers to any cell, prokaryotic or eukaryotic, including plant, yeast, worm, insect and mammalian. Mammalian cells include, without limitation; primate, human and a cell from any animal of interest, including without limitation; mouse, hamster, rabbit, dog, cat, domestic animals, such as equine, bovine, murine, ovine, canine, feline, etc. The cells may be a wide variety of tissue types without limitation such as; hematopoietic, neural, mesenchymal, cutaneous, mucosal, stromal, muscle spleen, reticuloendothelial, epithelial, endothelial, hepatic, kidney, gastrointestinal, pulmonary, T-cells etc. Stem cells, embryonic stem (ES) cells, ES-derived cells and stem cell progenitors are also included, including without limitation, hematopoeitic, neural, stromal, muscle, cardiovascular, hepatic, pulmonary, gastrointestinal stem cells, etc. Yeast cells may also be used as cells in this invention.

Cells also refer not to a particular subject cell but to the progeny or potential progeny of such a cell because of certain modifications or environmental influences, for example differentiation, such that the progeny mat not, in fact be identical to the parent cell, but are still included in the scope of the invention.

The cells used in the invention can be cultured cells, e.g. in vitro or ex vivo, as shown in the Examples herein. For example, cells cultured in vitro in a culture medium and environmental stimuli can be added to the culture medium. Alternatively, for ex vivo cultured cells, cells can be obtained from a subject, where the subject is healthy and/or affected with a disease. Cells can be obtained, as a non-limiting example, by biopsy or other surgical means know to those skilled in the art.

Cells used in the invention can present in a subject, e.g. as part of an in vivo assay. For the invention on in vivo cells, the cell is preferably found in a subject and an environmental stimuli is administered the subject. Methods for assaying bioluminescence are described in Tannous et al, (2005) Mol Therapy, 11 ;435-443 and other methods known to persons skilled in the art.

As used herein, the term “subject” is intended to include human and non-human animals. The term “non-human animals” includes all vertebrates, e.g. mammals, non-mammals, such as non-human primates, sheep, dog, cow, chickens, amphibians, reptiles etc. In certain embodiments, the subject is mammal, e.g., a primate, e.g., a human.

In order for a particular cell to express the proteins encoded by nucleic acid sequences, the nucleic acid must be introduced into the cell. Methods to introduce DNA into cells, the cell must be transformed by an appropriate vector. “Transformation”, as used herein, refers to the introduction of heterologous polynucleotide or nucleic acid sequence or fragment thereof into a host cell, irrespective of the method used, for example direct uptake, transfection or transduction. The present invention, therefore also relates to cells which have been transformed by at least one nucleic acid construct, wherein one construct comprises the sequence for Gaussia luciferase fluorescent conjugate protein of the present invention and expresses the Gaussia luciferase-fluorescent conjugate protein. The construct may be introduced into the cell by multiple means known to persons skilled in the art, including vectors, viral vectors, and non-viral means. Non-viral means include without limitation, fusion, electroporation, biolistics, transfection, lipofection, protoplast fusion, calcium phosphate transfection, microinjection, pressure-forced entry, naked DNA etc. or any other means known any person skilled in the art.

The term “vectors” used interchangeably with “plasmid” refer to a nucleic acid molecule capable of transporting another nucleic acid to which it has been linked. Vectors capable of directing the expression of genes and/or nucleic acid sequence to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. Other expression vectors can be used in different embodiments of the invention, for example, but are not limited to, plasmids, episomes, bacteriophages or viral vectors, and such vectors may integrate into the host's genome or replicate autonomously in the particular cell. Other forms of expression vectors known by those skilled in the art which serve the equivalent functions can also be used. Expression vectors comprise expression vectors for stable or transient expression encoding the DNA.

In one embodiment, the expression vector used herein are replicable DNA constructs which the nucleic acid is operatively linked to a suitable regulatory sequence capable of affecting the expression of the Gaussia luciferase-fluorescent protein conjugate. The construct may be introduced by homologous recombination, where it is desired that a construct be integrated at a particular locus. For example, one can delete and/or replace an endogenous gene at the same locus or elsewhere with the nucleic acid construct of this invention. For homologous recombination, the nucleic acid is cloned into specific vectors, including but not limited to; Ω or O-vectors, see, for example, Thomas and Capecchi, cell, (1987), 51;503-512, Mansour et al, nature, (1988) 336;348-352; and Joyner et al, nature (1989) 338;153-156.

Vectors comprising useful elements such as bacterial or yeast origins of replication, selectable and/amplifiable markers, promoter/enhancer elements for the expression in prokaryotes or eukaryotes, and mammalian expression control elements etc, may be used to prepare stocks of nucleic acid constructs and for carrying out transfections are well known in the art, and many are commercially available.

The term “viral vectors” refers to the use as viruses, or virus-associated vectors as carriers of the nucleic acid construct into the cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g EPV and EBV vectors.

Methods to induce a fluorescence signal require the excitation of the fluorescent protein at a particular wavelength to cause fluorescence excitation of the particular fluorescent protein. Detection of this fluorescence signal required. Methods to measure fluorescence may be carried out for example by fluorimetry, FACS and by microscope techniques well known by one skilled in the art. In this manner localization and/or quantification of the fluorescent protein may be determined.

The term “bioluminescence” as used herein is a type of chemiluminescence, refers to the emission of light by biological molecules, particularly protons. Essential conditions for bioluminescence comprise; molecular oxygen, either bound or free in the presence of an oxygenase; a luciferase, which acts on a substrate luciferin. The bioluminescence reaction is an energy-yielding chemical reaction in which a specific chemical substrate, a luficerin, undergoes oxidation, catalyzed by an enzyme, a luciferase.

The term “luciferase” refers to enzymes that catalyze the oxidation of luciferin, emitting light and releasing oxyluciferin. The term “bioluminescence-system” refers to the components that are necessary and sufficient to generate bioluminescence. These include luciferase, luciferins and any necessary co-factors or conditions. Virtually any bioluminescent system known to those skilled in the art will be amenable to use in the methods described herein.

The term “bioluminescence substrate” as used herein refers to the compound that is oxidized in the presence of luciferase and any necessary activators, and generates light. These substrates are referred to “luciferins” herein, and are substrates that undergo oxidation in a bioluminescence reaction. These bioluminescence substrates include any luciferin or analogue thereof, or any synthetic compound that generates light. Such molecules include naturally-occurring substrates, modified forms thereof and synthetic substrates [see e.g. U.S. Pat. Nos. those described in U.S. Pat. Nos. 5,374,534 and 5,098,828]. Exemplary luciferins include those described in U.S. Pat. No. 6,436,682, and derivatives thereof, analogues thereof, synthetic substrates, as well as dioxetanes [see e.g. U.S. Pat. Nos. 5,004,565 and 5,455,357], and other compounds that are oxidized by luciferase in a light-producing reaction [see, e.g. U.S. Pat. Nos. 5,374,534, 5,098,828 and 4,950,588]. Such substrates may be identified empirically by selecting compounds that are oxidized in bioluminescent reactions. Bioluminescence substrates, thus, include those compounds that those skilled in the art recognize as luficerins. In one embodiment, the luciferin is coelenterazine and analogues thereof, which include molecules in U.S. Pat. No. 6,436,682.,and for example, see Zhao et al, (2004), Mol Imaging, 3;43-54.

Methods to measure bioluminescence are well known to those skilled in the art. Bioluminescence reactions are also well-known to those skilled in the art, and any such reaction may be adapted for used in combination with articles of manufacture as described herein.

In another embodiment, bioluminescence can be measured by Bioluminescence Resonance Energy Transfer (BRET) (Boute et al, (2002) Trends. Pharmacol. Sci. 23;351-354; Morin & Hastings (1971) J Physioll 77;313-18) refers to the natural phenomena whereby green bioluminescence emission observed in vivo was shown to be the result of the luciferase non-radioactively transferring energy to an accessory green fluorescent protein (GFP). Energy transfer between two flurescent proteins (FRET) as a physiological reporter has been reported (Miyawaki et al, 1997, Natuew, 388;882-7). A similar reported is possible with a luciferase-GFP pair (see, e.g. U.S. Pat. No. 6,436,682 which is incorporated in its entirety herein for reference).

The term “environmental stimuli” refers to any entity which is normally not present or not present at the levels being administered in the cell. Environmental stimuli may be selected from a group comprising; chemicals; an action; nucleic acid sequences; proteins; peptides; or fragments thereof. A nucleic acid sequence functioning as an environmental stimuli may be RNA or DNA, and may be single or double stranded, and can be selected from a group comprising; nucleic acid encoding a protein of interest, oligonucleotides, PNA, etc. Nucleic acid sequence encoding environmental stimuli can also inhibit the activity of a regulatory sequence and/or constitutively active promoter. Such nucleic acid sequences include, for example, but not limited to, nucleic acid sequence encoding proteins that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof, functioning as an environmental stimuli can be any protein of interest, for example, but not limited to; mutated proteins; therapeutic proteins; truncated proteins, wherein the protein is normally absent or expressed at lower levels in the cell. Proteins of interest can be selected from a group comprising; mutated proteins, genetically engineered proteins, peptides, synthetic peptides, recombinant proteins, chimeric proteins, antibodies, humanized proteins, humanized antibodies, chimeric antibodies, modified proteins and fragments thereof. The environmental stimuli may be applied to the media, where it contacts the cell and induces its effects. Alternatively, the environmental stimuli may be intracellular within the cell as a result of introduction of the nucleic acid sequence into the cell and its transcription resulting in the production of the nucleic acid and/or protein environmental stimuli within the cell. An environmental stimulus also includes to any action and/or event the cells are subjected to. As a non-limiting examples, an action can comprise any action that triggers a physiological change in the cell, for example but not limited to; heat-shock, ionizing irradiation, cold-shock, electrical impulse, light and/or wavelength exposure, UV exposure, pressure, stretching action, fluorescence exposure etc. Environmental stimuli also include intrinsic environmental stimuli defined below. The exposure to environmental stimuli may be continuous or non-continuous.

The term “compound of interest” refers to any chemical, entity or moiety, including without limitation synthetic and naturally-occurring non-proteinaceous entities. In certain embodiments the compound of interest is a small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The term “intrinsic environmental stimuli” refers to any environmental stimulus which is intracellular to the cell. As a non-limiting example, the cell contains a mutated protein and/or dysfunctional biological pathway as a result of defect in the genome of the cell. As a non-limiting example, the defect may be a mutation and/or polymorphisms, and/or single nucleotide polymorphism (SNP). The defect may be known or unknown. For example, cells are from a patient affected with a disease (see; Example 1) or from cell lines generated from a patient and/or cell lines exhibiting a particular disease characteristic.

### General Methods of the Assay

The method involves the introduction into a cell of a nucleic acid construct encoding a secretable form of a luciferase enzyme conjugated to a fluorescent protein which is operatively linked to a regulatory sequence. In one embodiment, the luciferase is from the Gaussia genus. In another embodiment, a Gaussia luciferase-fluorescent fusion protein is used, and in another embodiment Gaussia luciferase-fluorescent protein conjugates are used. The introduction of the nucleic acid can be by any method described above.

Cells from any species and any tissue can be used to carry out the methods of the invention. The cells are cultured or maintained in a conventional culture medium under suitable conditions permitting growth of the cells. For example, the cell is cultured in standard tissue culture media containing the necessary reagents to select for cells which stably retain the nucleic acid construct described above. Cells may be cultured in standard tissue culture dishes e.g. multidishes and microwell plates, or in other vessels, as desired. In some configurations, the assay can be conducted in a 96 well; 386-well or other multi-well plates.

In one embodiment, environmental stimuli are added to the culture media to assess their effect on specific biological systems within the cell. Cells at this time may be bathed in tissue culture media (with or without serum) or balanced salt solution. The environmental stimuli may be applied to the media, wherein it contacts the cell and induces its effects or it may be intracellular within the cell. In one embodiment, the environmental stimuli may be a chemical or library of chemicals. These may be cell permeable and therefore added directly to the cells. Alternatively, it may be necessary to make the cells permeable using streptolysin O, tetanolysin or another permeabilizing agent known by persons skilled in the art. In another embodiment, the environmental stimuli may be a protein or peptide or a nucleic acid sequence. These may be directly applied to the cells, or alternatively introduced into the cells by a vector and when expressed, these proteins and nucleic acids act as intracellular environmental stimuli. In one embodiment, the environmental stimuli that are nucleic acid sequences are individual DNA molecules having one or more genes, each operatively linked to the same or different regulatory sequences to the one operatively linked to the Gaussia luciferase-fluorescent conjugate protein. The constructs may be introduced simultaneously, or consecutively, each with the same or different markers. In an alternative embodiment, the nucleic acid sequences encoding the environmental stimuli are present on the nucleic acid construct with the Gaussia luciferase-fluorescent conjugate protein operatively linked to the regulatory sequence.

In another embodiment, the environmental stimuli is an intrinsic environmental stimuli, for example, the cell to which the nucleic acid construct is introduced is a cell from a cell line which has biological properties of a particular disease characteristic, and/or the cell was from a biopsy of a patient with a disease, and carries a mutated protein. In another example, the cell is a genetically engineered cell in which one or more of the environmental stimuli are intracellular.

In some embodiments, the cells are exposed to one environmental stimuli. In other embodiments, the cells are exposed to more than one environmental stimuli. The environmental stimuli may be of the same type (i.e. chemical, procedure, protein, nucleic acid, intrinsic stimuli etc.) or may be any combination of environmental stimuli. Exposure of the cells to environmental stimuli may be simultaneous, sequentially or consecutive, and in any order. Cells may be exposed to any environmental stimuli once or any number of times. The time of exposure of the cells to the environmental stimuli can vary, depending on the environmental stimuli, the cells being used and the biological system being assessed in the method of the invention.

During incubation and growth of the cells in the presence and absence of environmental stimuli, and depending on the regulatory sequence used, the cell is capable of expressing the Gaussia luciferase-fluorescent conjugate protein, comprising steps of gene transcription, translation and post-translational modification. The expressed Gaussia luciferase-fluorescent conjugate protein is processed through the secretory pathway and secreted into the culture media.

The expression of the Gaussia luciferase-fluorescent conjugate protein can be assessed by fluorescence and/or by bioluminescence. In one embodiment, fluorescence is used as a qualitative and/or quantitative measure of the expression and processing of the Gaussia luciferase conjugate protein through the secretory pathway in the cell, which can be measured by fluorescence detection methods, for example, fluorescence microscopy, FACS etc.

In another embodiment, bioluminescence is used as a measure of expression and secretion of the Gaussia luciferase conjugate protein into the culture media. In one embodiment, a luciferin, for example but not limited to, coelenterazine and its analogues is added to the culture media, and the bioluminescence monitored. In another embodiment, the media is collected and assayed for bioluminescence. In such an embodiment, the cells can be repeatedly assessed for luciferase activity. Further quantitation of secretion can easily be monitored by taking aliquots of the medium conditioned by living cells over time. Since the cells are not disrupted during assay they can be used for other assays in parallel. Further, since the cells are not disrupted, conditioned media can be sampled repeatedly for time course studies from a single well and cells can be used for further studies, such as RNA or protein analysis.

Since the Gaussia luciferase is naturally secreted, the assay is performed on small samples of the conditioned media, with no need to lyse the cells, which makes it much faster and more convenient than assays with other luciferases such as firefly luciferase (FLuc) which is used in the SUPERLIGHT™ luciferase reporter gene assay (Bioassays, CA) where cell lysis is required. The present invention is advantageous over other systems in that the methods serve to reduce the variability of transfection efficiency when different wells are used. Further, since the Gaussia luciferase is over two thousand fold more sensitive than firefly luciferase (FLuc) or Renilla luciferase (Tannous et al., 2005), it can easily be used to measure promoters over a wide range of activities with no need for signal enhancement for promoters with low activity.

### Applications

Monitor protein transit through the secretory pathway. In one embodiment, the assay can be used to monitor the transit of proteins through secretory pathway. In such an embodiment, the Gaussia Luciferase (GLuc) fluorescent conjugate protein is operatively linked to a constitutive promoter, and on expression, the subcellular localization of the GLuc-fluorescent conjugate protein can be visualized for qualitative assessment and its level of secretion quantified for a quantitative measure of secretion. In such an embodiment, the fluorescent protein enables monitoring of the primarily entry of the protein conjugate into the endoplasmic reticulum (ER), and co-staining with ER markers such as translocon markers, for example but not limited to ribophorin and Sec61, enable assessment of the punctuate localization of the GLuc-fluorescent conjugate indicating its localization in the ER. Thus the methods of the present invention using such a Gluc-fluorescent protein conjugate reporter has the advantages of marking a distinct position in the secretory pathway, namely the ER entry points, and being the most sensitive assay to date in living cells.

In a related embodiment, the methods of this invention can be used to study environmental stimuli that disrupt and/or modify the normal functioning of the secretory pathway, for example but not limited to the effect of mutated proteins (see Example 1).

Environmental stimuli that cause a reduction in the bioluminescence signal and/or abnormal localization of the GLuc-fluorescent conjugate protein in the cell, observed by fluorescence visualization compared to control cells (i.e. cells not subjected to the environmental stimuli) can be identified as environmental stimuli that have a negative effect and/or disrupt the secretory pathway. Furthermore, the methods of this invention can be used to screen for, identify and study stimuli that affect the secretion of proteins, for instance, but not limited to, environmental stimuli which prevent and/or restore the function of a dysfunctional secretory pathway. Accordingly, environmental stimuli that increases bioluminescence and/or restore normal localization of the GLuc-fluorescent conjugate protein may be identified to improve secretory pathway function.

Regulatory sequence Assay. In another embodiment, the methods of the invention can used in the assessment of promoter activities and/or regulatory sequences. In one embodiment, the effects of modification to a promoter or regulatory sequence can be assessed, as an exemplary example, mutations, polymorphisms, single nucleotide polymorphisms (SNPs), truncations, methylations etc. These modifications can be assessed in the presence or absence of environmental stimuli. In another embodiment, the methods can be used to assess the response of a promoter and/or regulatory sequences to different environmental stimuli, for example but not limited to; drugs, compounds; ionizing radiation; proteins; nucleic acids etc. In another embodiment, the methods can be used to study specific regulatory elements within the regulatory sequences that are induced or inhibited in response to specific environmental stimuli. In such an embodiment, environmental stimuli or modifications in the regulatory sequence that result in an increase in the bioluminescence signal and/or fluorescence signal compared to control cells and/or sequences, may be identified to activate regulatory sequence which normally induce gene expression (e,g., promoter or enhancer sequences) and inhibit regulatory sequences which normally repress gene expression (such as a repressor sequences etc). Conversely, environmental stimuli that result in a reduction in the bioluminescence signal and/or fluorescence signal compared to control cells, may be identified to inhibit regulatory sequence which normally induces gene expression (e,g., promoter or enhancer sequences) and activate regulatory sequences which normally repressed gene expression (such as a repressor).

Accordingly, since the cells are not disrupted, conditioned media can be sampled repeatedly for time course studies from a single well and the cells can be used for sequential and subsequent further studies, such as RNA and/or protein analysis. Further, since the Gaussia luciferase is over two thousand fold more sensitive than other luciferases, such as firefly luciferase (FLuc) or Renilla luciferase (Tannous et al., 2005), it can easily be used to measure promoters over a wide range of activities with no need for signal enhancement for promoters with low activity.

In another embodiment, the Gluc-fluorescent protein conjugate can be used to monitor libraries to promoters. In another embodiment, the cells containing the Gluc-fluorescent protein conjugate under the control of a specific regulatory element can be used to monitor the effect of modifications to regulatory sequences. Modifications include, for example, but not limited to, mutations, truncations, polymorphisms, SNPs, methylations and/or fragments of the regulatory sequences. In such an embodiment, one or more modifications to the regulatory element alone may be assessed, and/or the effect of the modification in response to a specific environmental stimuli.

Monitor transcriptional activation. In another embodiment, the methods of the invention can be used to study transcriptional activation in cells. In such an embodiment, the regulatory sequences operatively linked to the Gauissa luciferase fluorescent conjugate protein are responsive to particular transcription factors and regulatory proteins. As a non-limiting example, the regulatory sequences are promoters that are activated by specific transcription factors. In a related embodiment, the methods of this invention can be used to study environmental stimuli that affect the activity of the regulatory sequence. As an illustrative but non-limiting example, the effect of environmental stimuli, such as a procedure, (e.g. ionizing irradiation) on the activity of a regulatory sequence can be assessed. Further, the methods can also be used to assess the effect of a second environmental stimuli on the activity of promoter, for example, environmental stimuli (e.g. compounds) which affect the activity of the regulatory sequence in response to the first environmental stimuli (e.g ionizing irradiation).

As an exemplary example, the NFκB promoter can be used as the regulatory sequence operatively linked to nucleic acid encoding the Gaussia luciferase fluorescent conjugate protein. The transcriptional activity of the NFκB promoter can be assessed in response to environmental stimuli such as irradiation by assessing bioluminescence in the media and/or fluorescence in the cell. An increase in bioluminescent indicates transcriptional activity of the NFκB promoter. The assessment of bioluminescence in the media and/or fluorescence in the cell after the exposure of cells to another environmental stimuli (e.g. radiometric compounds), either simultaneous with the first environmental stimuli or subsequently, enables the assessment of the second environmental stimuli on the activity of the NFκB promoter. Accordingly, a decrease in bioluminescence identifies environmental stimuli that reduce the activity of the regulatory sequence, in this case the NFκB promoter, and this enables screening for efficacy of radiometric compounds.

In another embodiment of the invention can be used to screen for, identify and study stimuli that affect the transcriptional activity of regulatory sequences, for instance, but not limited to, environmental stimuli which activate or inhibit transcriptional activation and/or stimuli that have transcriptional modulating activity of the specific regulatory sequence.

Monitor signaling pathways. In another embodiment, the methods of the invention can be used to study signaling pathways in cells. In such an embodiment, the regulatory sequences operatively linked to the Gauissa luciferase fluorescent conjugate protein are responsive to particular signaling molecules. As an exemplary example, the regulatory sequence can be responsive to a protein kinase signaling molecule, as a non-limiting example, PKC, and the regulatory sequence is operatively linked to nucleic acid encoding the Gaussia luciferase fluorescent conjugate protein. The activation of the signaling molecule will activate or inhibit the regulatory sequence and can be detected by bioluminescence and/or fluorescence. The activity of the signaling molecule can be assessed in response to environmental stimuli by assessing changes in bioluminescence in the media and/or fluorescence in the cell. For example, activation of NFκB transcription factor and early growth factors responsive (Egr-1) factor, as well as p53 apoptosis induction in response to environmental stimuli.

5. Monitor cell viability. In another embodiment, the methods of this invention can be used to monitor cell viability and cell death. In this invention, the secretion of Gaussia luciferase-fusion protein is proportion to the cell number, therefore the level os bioluminescence can be used as a method to measure the number of cells. Accordingly, in such an embodiment, the Gaussia luciferase-fluorescent protein conjugate is operative linked to a regulatory sequence encoding a constitutively active promoter. In one embodiment of the invention relates to methods monitor the viability of cells by bioluminescence in response to environmental stimuli. In one embodiment, the environmental stimuli can be intrinsic environmental stimuli. Accordingly, the methods of this invention can be used to monitor cell death over time in the same well by repeated assay of the conditioned media for Gluc activity, since as cells die, the synthesis and secretion of the Gaussia luciferase-fluorescent conjugate protein is attenuated. In such an embodiment, a decrease in the bioluminescence signal compared to control cells indicates an increase in cell death as the cells are dye and there are less cells present, whereas an increase in bioluminescence indicates cell viability and in some instances a possible increase in cell proliferation. In one embodiment, the methods of the invention are done on proliferating cells. In another embodiment, the methods of the invention are done on non-dividing cells, for example terminally differentiated cells or cell that have undergone mitotic arrest, for example with treatment with mitomycin-c or other mitotic altering agents known to persons skilled in the art. The cells do not need to be disrupted by the sampling procedure, so the Gluc-fluorescent protein conjugate viability assay has an advantage over the firefly luciferase (FLuc) cell viability assay (Bioassays) in that no cell-lysis is required. The Gluc-fluorescent protein conjugate viability assay also has an advantage over other apoptosis detection assays, such as CELLQUANTI-MTT™ cell Viability assay kits (Bioassays) as in those cases the reagents need to be added to the cells, which makes it impossible to do time course measurements in the same well.

Monitor protein-protein interactions. In another embodiment, the methods can be used to study protein-protein interactions in cells by use of BRET (bioluminescence-Energy Transfer system) (see Boute et al (2002) Trends Pharmacol. Sci. 32;351-354) where the Gaussia luciferase and the fluorescence protein of the conjugate are in close proximity to each other (100 Å) the energy resulting from chemically excited state of luciferase-luciferin complex would be transferred to the fluorescent protein, which in turn, would re-emit the light in a narrow wavelength according to the fluorescent protein present (see Tsien et al (2003) Nat. Rev. Mol. Cell. Biol. Suppl. Ss16), and also increasing the yield of the bioluminescence of the luciferase (see Szent-Gyorgyi (1999) Mol. Imaging: Reporters, Dyes, markers and instrumentation, San Jose, Calif. Proc. SPIE, 3600;4-11).

### Alternative Applications:

In a further embodiment, the methods can be used in parallel assays, to enable the monitoring of two biological processes simultaneously in the same cell and/or same subject. The methods described herein can be combined for sequential imaging of two luciferases with the other luciferases having different luminescence kinetics (see Shah et al (2003) Oncogene 22; 6865-6872 and Bakaumik et al (2002) Proc. Natl. Acad. Sci. U.S.A. 99;377-382; Tannous et al, (2004) Mol Therapy, 11 ;435-443).

In another embodiment, the Gaussia luciferase nucleic acid sequence can be substituted for any secretable luciferase or luciferase photoprotein enzyme.

In another embodiment, the methods described herein can be used in vivo, such methods are described in see Shah et al (2003) Oncogene 22; 6865-6872 and Bakaumik et al (2002) Proc. Natl. Acad. Sci. U.S.A. 99;377-382; Tannous et al, (2004) Mol Therapy, 11;435-443).

### Cells

The cells used in the methods described here in may be prokaryotic, but are preferably eukaryotic, including plant, yeast, worm, insect and mammalian. In another embodiment, the cells are mammalian cells, particularly primate, human and can be associated with any animal of interest, including but not limited to domestic animals, such as equine, bovine, murine, ovine, canine, feline, etc. Among these species, various types or cells can be used, such as hematopoetic, nueral, messencymal, cutaneous, mucosal, stromal, muscle spleen, reticuloendothelial, epithelial, endothelial, hepatic, kidney, gastrointestinal, pulmonary etc. Also, in another embodiment, the cells are stem cells and progenitors, such as hematopoeitic neural, stromal, muscle, hepatic, pulmonary, gastrointestinal, etc.

In one embodiment, the method can be used on cultured cells, e.g. in vitro or ex vivo, as shown in the Examples herein. For example, cells can be cultured in vitro in culture medium and the contacting step can be achieved by adding one or more environmental stimuli to the culture medium. Alternatively, the method can be performed on cells present in a subject, e.g. as part of an in vivo therapeutic protocol. For in vivo methods, the cell is preferably found in a subject and contacting of the environmental stimuli with the cell is achieved by administering the environmental stimuli to the subject. As used herein, the term “subject” is intended to include human and non-human animals. The term “non-human animals” includes all vertebrates, e.g. mammals, non-mammals, such as non-human primates, sheep, dog, cow, chickens, amphibians, reptiles etc. In certain embodiments, the subject is mammal, e.g., a primate, e.g., a human.

In another embodiment, the methods of the invention can be performed in a cell-free system prepared from cells as described above, or from the purified components in lieu of engineered cells. In a cell-free implementation, Gaussia luciferase-fluorescent protein conjugate may be produced from the nucleic acid construct by synthetic or non-synthetic peptides, peptides or small molecules identified from cell broths, natural extracts etc. The method of the assays is the same as above, except that it is performed in a cell-free environment. In this case, the Gaussia luciferase fluorescent protein conjugate is still operatively linked to the regulatory sequence.

In another embodiment, the methods of the invention can be used for in vitro and/or in vivo monitoring tumor size. The term “tumor” refers to any tissue mass or tissue type or cell type that is undergoing uncontrolled proliferation. The assessment of tumor size can be performed by monitoring bioluminescence by imaging (for example, Tannous et al, 2005, Mol Therapy, 11;435-443) or measuring Gluc activity from any biological sample taken from the subject with the tumor, selected from a list comprising; tumor biopsy, blood, serum etc. In such an embodiment, the methods can be used to monitor tumor physiology before, during or after anti-cancer therapies, for example but not limited to, monitoring tumor growth (increase in size), tumor reduction (decrease in size), malignancy, tumor migration or spreading, tumor regression, etc.

In one embodiment, the nucleic acid constructs may be introduced as single DNA molecules containing all the genes for the Gaussia-luciferase florescent protein conjugate which is operatively linked to the regulatory sequence, or alternative embodiments, involve introducing the nucleic acid construct as different DNA molecules having one or more genes, each operatively linked to the regulatory sequence. The constructs may be introduced simultaneously, or consecutively, each with the same or different markers.

The systems and cells provided herein can be used for high throughput screening protocols, intracellular assays, medical diagnostic assays, environmental testing and biological research assays, and appropriate systems known by persons skilled in the art. This invention further provides kits for the foregoing applications. Such kits contain DNA constructs encoding and capable of directing the expression of the Gaussia luciferase fluorescent protein conjugate of this invention, and in some embodiments involving directed nucleic acid transcription of the Gaussia luciferase fluorescent protein conjugate operatively linked to one or more regulatory sequences of interest. Alternatively, the nucleic acid construct may contain a multiple cloning site for insertion of a particular regulatory sequences of interest desired by the practitioner. Such kits may also contain a sample to nucleic acid sequence operatively linked to a constitutively active promoter as a control.

The practice of the present invention employs, unless otherwise indicated, conventional techniques of cell biology, cell culture, molecular biology, transgenic biology, microbiology, recombinant DNA and immunology, which are within the skill of the art. Such techniques are explained fully in the literature. See, for example, Molecular Cloning A Laboratory Manual, 2^{nd} Ed., ed. By Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis er al, U.S. Pat. No: 4,683,195; Nucleic Acid Hybridization (B. D. Hames & S. J. Higgins eds. 1984); Transcription and Translation (B. D. Hames & S. J. Higgins eds. 1984); Culture of Animal Cells (R. I. Freshley, Alan R. Liss, Inc., 1987); Immobilized cells and Enzymes (IRL Press, 1986); B. Perbal, A Practical Guide to Molecular Cloning (1984); the treatise, Methods in Enzymology (Academic Press, Inc., N.Y.); Gene Transfer vectors for Mammalian Cells (J. H. Miller and M. P. Calos eds., 1987, Cold Spring Harbor Laboratory); Methods in Enzymology, Vols. 154 and 155 (Wu et al, eds.), Immunochemical methods In Cell and Molecular Biology (Mayer and Walker, eds., Academic Press, London, 1987); Handbook of Experimental Immunology, Volumes I-IV (D. M Weir and C. C. Blackwell eds., 1986); Manipulating the Mouse Embryo (Cold Spring Harbor Press: Cold Spring Harbor, N.Y., 1986).

Additional background information and general guidance to the practitioner with respect to the design, assembly, incorporation into plasmid, and transfection of constructs for such protein conjugates or fusion proteins and regulatory sequences is available for the following published international patent applications: WO94/18317; WO95/02684; WO95/24419 and WO 96/41865 and the contents of which are incorporated herein by reference.

### Examples

There are many ways to utilize the Gluc-fluorescent protein conjugate assay. The following examples only illustrate particular ways to develop assays for to study particular biological systems, and should not be construed to limit the invention.

Methods.

Cell culture. The following fibroblast lines were generated from skin biopsies: human controls (HF6, HF18, HF19, HF24. HF60) and affected DYT1 ΔGAG carriers (HF47, HF48, FFF13111983, FFF076111984). HF lines were generated in our laboratory (Breakefield et al., 1981) and FFF13111983 and FFF076111984 were obtained from Dr. Mirella Filocamo (Inst. G. Gaslini, ITALY). All cells were grown in Dulbecco's modified Eagle medium (DMEM; Gibco, Rockville, Md.) supplemented with: 4.5 g/L glucose, 2 mm glutamine, 10% fetal bovine serum, 50 U/mL penicillin and 50 μg/mL streptomycin (Gibco). Cultures were maintained at 37° C. in 95% air/5% CO₂.

Vectors and expression cassettes. Lentivirus vectors were derived from a self-inactivating lentivirus, CS-CGW in which the transgene is under the cytomegalovirus (CMV) immediate early promoter followed by an internal ribosome entry site (IRES) and the GFP cDNA (Sena-Esteves et al., 2004). One set of vectors contained the cDNA for human wt or mt torsinA under the CMV promoter (Hewett et al., 2006). In addition a cDNA encoding humanized Gluc (Prolume Ltd./Nanolight) alone or fused in-frame at the carboxy terminal with enhanced YFP (Invitrogen, Carlsbad, Calif.) (SEQ ID No.5) was inserted downstream of the CMV promoter and the GFP cDNA was replaced with a cDNA for the optimized blue fluorescent protein, cerulean (Rizzo et al., 2004; from Dr. David Piston, Vanderbilt Univ. Med. Ctr., TN). The cDNA sequence encoding the Gaussia luciferase (Gluc) can be amplified by PCR using a pGluc (Nanolight) and primers (a) 5′-CTCGCGGAATTCAAAATGAAACCAACTGAA-3′(SEQ ID NO:6) and (b) 5′-AGCTGCCTCGAGGCATTAATCACCACCGGCACC-3′ (SEQ ID NO:7). Gaussia luciferase (Gluc) cDNA, codon optimized for mammalian cell expression (hGluc) was amplified by PCR using pcDNA-pGluc (Nanolight) and primers (c) 5′-CTCGCGGAATTCAAAATGGGAGTCAAAGTTCTG-3′ (SEQ ID NO:8) and (d) 5′-AGCTGCCTCGAGGCATTACTAGTCACCACCGGC-3′ (SEQ ID NO:9). All primers were designed to introduce an EcoRI site at the 5′ end and an XhoI site of the 3′ end of each cDNA. Vectors were produced by co-transfection of 293T cells with the lentivirus packaging plasmid (pCMVR8.91), envelope coding plasmid (pVSVG) and vector construct yielding typical titers of 10⁸ transducing units (tu)/ml (Sena-Esteves et al., 2004).

Gluc activity. To monitor Gluc secretion, cells were plated in 150 cm dishes (1.5 million cells/dish) and infected with lentivirus vector encoding Gluc (or Gluc-YFP) and cerulean at a multiplicity of infection (M.O.I.)=50. Seventy-two hrs post-infection, cells were re-plated in 12 well plates (2,5000 cells/well). Luciferase activity was monitored in conditioned, cell-free medium and in living washed cells at varying time points after replating. Activity was measured with a luminometer (Dynex) after adding 20 μM coelenterazine (Prolume Ltd./Nanolight) to the medium or cells, with photon signals integrated over 2 sec (Tannous et al., 2005).

Sensitivity of Gluc versus SEAP. 293T cells were plated in 60 mm dishes (5×10⁵ cells/dish) and co-transfected with a plasmid encoding Fluc under control of CMV promoter (pHGC-Fluc) [17] and either a plasmid encoding Gluc (pHGC-hGluc) [17] under the control of CMV promoter or a plasmid encoding secreted alkaline phosphatase (pSEAP2-control vector, Clontech) under control of SV40 early promoter using Lipofectamine (Invitrogen, Carlsbad, Calif.). Twenty-four h after transfection, cells were harvested, washed with PBS and different numbers of cells were plated in a well of 96 well plate. Twenty-four h later, cell-free conditioned medium were assayed for either Gluc or SEAP. Transfection efficiency was normalized to the level of Fluc activity in viable cells.

Luciferase activity. Gluc activity was measured by adding 20 μM coelenterazine (Prolume Ltd./Nanolight, Pinetop, Ariz.) to an aliquot of the cell-free conditioned medium and measured for 10 sec using a luminometer (Dynex, Richfield, Minn.). For Fluc detection, 450 μM Beetle _{D}-luciferin (Molecular Imaging Products, Ann Arbor, Mich.) was added directly to the viable cells, in a 96 well plate and measured as above. The signal was measured for 10 sec and integrated over 2 sec in both cases.

Secreted alkaline phosphatase (SEAP) assay. An aliquot of the conditioned cell-free medium was used to monitor the SEAP activity using the Great EscAPe SEAP kit (Clontech). Briefly, 15 μL cell free medium was mixed with 45 μL of 1× dilution buffer and incubated at 65° C. for 30 min. Samples were cooled down to room temperature and mixed with 60 μL assay buffer, incubated for 5 min at room temperature before adding 60 μL of chemiluminescent enhancer containing 1.25 mM CSPD substrate (3-(4-methoxyspiro{1,2-dioxetane-3,2′-(5′-chloro)-tricyclo[3.3.1.1.3,7]-decan}-4-yl) phenyl phosphate). After 15 min of incubation at room temperature, chemiluminescence was measured using a luminometer.

ER stress: total RNA isolation, reverse transcription and real-time qPCR. 293T cells were plated at 1×10⁶ cells/mL in a 12-well plate. Cells were treated with different concentrations of DTT (Sigma) for 4 h. Total RNA was isolated using the RNeasy mini kit (Quiagen, Valencia, Calif.) and concentration was determined by measuring the OD₂₆₀ using a spectrophotometer (Bio-Rad, Hercules, Calif.). Reverse transcription was performed on 200 ng RNA (in 10 μl) at 37° C. for 60 min using an Omniscript reverse transcription kit (Quiagen). Two μL of each cDNA sample were used for real-time PCR. Biological triplicates were measured twice. The following primers were used: Forward 5′-GGTCTGCTGAGTCCGCAGCAGG-3′ (SEQ ID NO: 10) and Reverse 5′-GGGCTTGGTATATATGTGG-3′ (SEQ ID NO: 11). These primers were designed to span a 26 bp intron in the unspliced XBP1 mRNA [12]. For normalization, we used human GAPDH primers: Forward 5′-TGGAAAGCTGTGGCGTGATGGCCG-3′ (SEQ ID NO: 12) and Reverse 5′-CACCCAGAAGACTGTGGATGGCCCCT-3′ (SEQ ID NO: 13). Real-time PCR was performed in an ABI PRISM 7000 Sequence Detection System Thermal Cycler (Applied Biosystems, Foster City, Calif.) in a total volume of 30 μL, using the SYBR green PCR master mix (Applied Biosystems) with 10 pMoles of the primers set. The fold increase was calculated based on Ct values of treated cells relative to control non-infected and non-treated cells normalized to the values for GAPDH endogenous control.

For real-time monitoring of ER-stress with Gluc, 293T cells infected with lentivirus vector carrying the expression cassette for Gluc. Forty-eight h later, cells were treated with different concentration of DTT and aliquots of the conditioned medium were assayed for Gluc activity at different time point as above.

Western blot. Total cell lysates were prepared in lysis buffer containing 150 mM NaCl, 50 mM TRIS, pH 8.0, 1% NP-40, 0.5% deoxycholate, 0.1% SDS, and protease inhibitors (PI Complete; Boehringer Mannheim, Indianapolis, Ind.). Forty μg protein were electrophoresed in 12.5% SDS-polyacrylamide gels, and transferred to nitrocellulose membranes (Bio-Rad). Membranes were blocked overnight in 10% non-fat milk powder in TBST (150 mm NaCl, 50 mm TRIS, pH 7.9, 0.5% TWEEN) and probed with antibodies against Gluc (1:500, prepared by the Neuroscience Center Monoclonal Antibody Production Core at Mass. General Hospital) or BiP (Stressgen, College, Pa.) (1:200), diluted in TBST. Membranes were then incubated with horseradish peroxidase (HRP) conjugated to secondary antibodies: sheep anti-mouse IgG-HRP (1:10,000) or donkey anti-rabbit IgG-HRP (1:10,000) (Amersham Pharmacia Biotech, Piscataway, N.J.). For protein detection we used SuperSignal West Pico Chemiluminescent Substrate™ (Pierce, Rockford, Ill.).

Blocking of secretory pathway. 293T cells were infected with the lentivirus vector expressing Gluc. Forty-eight h post-infection, cells were treated with either 5 μg/ml BFA, 3 μg/ml monensin A (MonA), 10 μg/ml nocodazole (Noc) or 5 μg/ml cytochalasin B (CytB) all obtained from Sigma. Twenty-four h later, the Gluc activity was measured in the conditioned medium as above.

Immunocytochemistry. HF8 human fibroblast cells were plated on coverslips (100 cells/coverslip) and control or BFA treated cells (for 24 h) were extracted with digitonin. Cells were then fixed with 4% paraformaldehyde in PBS for 10 min at room temperature, washed with PBS and incubated with 0.1% NP-40 in PBS for 10 min. Blocking was performed using 10% goat serum (Vector Laboratories, Burlingame, Calif.) in PBS for 1 h. Cells were incubated with monoclonal mouse anti-Gluc antibody (1:100) and PDI (Stressgene, 1:600), for 1 h at 37° C. For fluorescence detection we used secondary antibodies, conjugated to Cy3 affiniPure donkey anti-mouse (1:1000; Jackson Immuno Labs, West Grove, Pa.) or Alexa 488 goat anti-rabbit (1:2000; Invitrogen-Molecular Probes, Carlsbad, Calif.). Coverslips were mounted onto slides using gelvatol mounting medium containing 15 μg/ml anti-fade agent 1,4-diazabicyclo(2.2.2)-octane (Sigma). Images were captured using an inverted fluorescent microscope (Nikon TE 200-U) coupled to a digital camera. In some experiments, Cells were grown on coverslips and fixed with 4% paraformaldehyde in PBS for 20 min at room temperature. After thorough rinsing with PBS, coverslips were incubated with 0.1% NP-40 in PBS for 20 min, followed by blocking with 10% goat serum (Vector Laboratories, Burlingame, CA) in PBS for 1 hr. Cells were incubated with antibodies to torsinA (1:1000), PDI (1:600), Sec61α (1:200), ribophorin I (1:100), and/or COPII (1:100), for 1 hr at 37° C. Coverslips were washed with PBS and incubated with secondary antibodies, conjugated to Cy3 affiniPure donkey anti-mouse, (1:1000; Jackson Immuno Labs, West Grove, Pa.) or Alexa 488 goat anti-rabbit (1:2000; Molecular Probes) for 1 hr at 37° C. Coverslips were mounted onto slides using gelvatol mounting medium containing 15 μg/ml anti-fade agent 1,4-diazabicyclo(2.2.2)-octane (Sigma). Images were captured using an inverted fluorescent microscope (Nikon TE 200-U) coupled to a digital camera.

Antibodies. Antibodies used in this study were to the following proteins: torsinA (D-M2A8; Hewett et al., 2003); α-tubulin (DM1A; Sigma, St Louis, Mo.); glyceraldehyde-3-phosphate dehydrogenase (GAPDH; Chemicon, Temecula, Calif.); GFP (Molecular Probes Inc., Eugene, Oreg.), protein disulfide isomerase (PDI; SPA-891; Stressgen, Ann Arbor, Mich.), ribophorin I (Santa Cruz Biotechnology, Santa Cruz, Calif.), Sec61α (Affinity BioReagents, Golden, Colo.), COPII (Affinity Bioreagents), ubiquitin (Stressgen), and calnexin (Stressgen).

Western blot. Cell lysates were prepared by washing the cells twice with PBS, and resuspending the cell pellet in lysis buffer containing 150 mM NaCl, 50 mM TRIS, pH 8.0, 1% NP-40, 0.5% deoxycholate, 0.1% SDS, and protease inhibitors (PI Complete; Boehringer Mannheim, Indianapolis, Ind.). Protein concentrations were determined using the Coomassie plus protein assay (Pierce, Rockford, Ill.) and a bovine serum albumin standard (Bio-Rad, Hercules, Calif.). Proteins were resolved by electrophoresis in 12.5% polyacrylamide gels, transferred electrophoretically to nitrocellulose membranes (Bio-Rad) and stained for protein with 0-2% Ponceau S (Sigma), as described (Hewett et al., 2006). Membranes were blocked overnight in 10% non-fat milk powder in TBST (150 mm NaCl, 50 mm TRIS, pH 7.9, 0.5% TWEEN) and probed with antibodies against GFP (1:2000), α-tubulin (1:10,000), GAPDH (1:4000), calnexin (1:5000), and torsinA (1:100) diluted in TBST and visualized with horseradish peroxidase (HRP) conjugated to secondary antibodies and SuperSignal West Pico Chemiluminescent Substrate™ (Pierce, Rockford, Ill.). Secondary antibodies for western blots were: sheep anti-mouse IgG-HRP (1:10,000) or donkey anti-rabbit IgG-HRP (1:10,000) (Amersham Pharmacia Biotech, Piscataway, N.J.).

Differential solubilization of cells. Proteins in different cell compartments were fractionaed by using digitonin to release soluble proteins and Triton X-100 as a non-ionic non-denaturing detergent to release ER proteins (Levine et al., 2005). Human fibroblast monolayer cultures (150 cm plates) were placed on ice and rinsed with PBS. Then a digitonin solution (150 μg/ml digitonin in 50 mM HEPES, pH 7.4, 100 mM KAc, 2.5 mM MgAc2) was added for 5 min and the lysate was collected. After rinsing 4× in PBS, extraction was carried out using a TritonX-100 solution (1% TritonX-100 in 50 mM, HEPES pH 7.4, 500 mM KAc, 5 mM MgAc2) for 5 min. Proteins in digitonin and TritonX-100 extracts were precipitated with 85% acetone. Remaining cell components were washed 3× with PBS and scraped off the plate using a rubber policeman in PBS. Protein concentrations were determined using the Coomassie plus protein assay (Pierce, Rockford, Ill.) and a bovine serum albumin standard (Bio-Rad, Hercules, Calif.). All samples were resolved by SDS polyacrylamide gel electrophoresis and western blotting.

Immunoprecipitation. For immunoprecipitations (IPs), human fibroblasts (2×10⁶) were lysed by resuspension in 1 ml ice-cold RIPA buffer (150 mM NaCl, 50 mM TRIS, pH 7.5, 1% NP-40. 0.5 deoxycholate, 0.1 SDS) for 30 min. Lysate supernatants were subjected to IP overnight at 4° C. using 7 μl D-M2A8 (anti-torsinA antibody) per 500 μl lysate and 3 mg/ml Protein-G agarose (Roche Applied Sciences), as described (Hewett et al., 2000) Beads were washed 3× with PBS and resuspended in SDS sample buffer for SDS-PAGE and western blotting.

### Example 1

Monitor the transit of proteins through secretory pathway. Gluc-YFP fusion protein is used as reporter protein to monitor processing through the secretory pathway. As an illustrative example, the processing of Gluc-YFP was assessed in fibroblasts from normal patients and from patients affected with DYT1, an early onset torsion dystonia that is a dominantly inherited movement disorder characterized by sustained, involuntary muscle contractions and abnormal posturing (Fahn, 1988). A specific mutation underlies most cases of DYT1 dystonia—a GAG deletion in the coding region of the DYT1 gene encoding torsinA which results in a loss of a glutamic acid residue (ΔE302/303) in the carboxy terminal region of the protein (Ozelius et al., 1997; Kabakci et al., 2004).

Levels of Gluc activity were measured in cells and media, and the intracellular location was visualized using a Gluc-yellow fluorescent protein (Gluc-YFP) fusion protein following infection of cells with a lentivirus vector encoding these reporters. Control cells secreted active Gluc in a linear manner with respect to time and cell number and showed an ER localization of Gluc-YFP. Patient cells were found to have a decrease in the rate of secretion of Gluc, as compared to control cells, with a substantial fraction of intracellular Gluc-YFP being in the cytoplasmic fraction. In both cell types immunoprecipitation of torsinA revealed an association with Gluc-YFP.

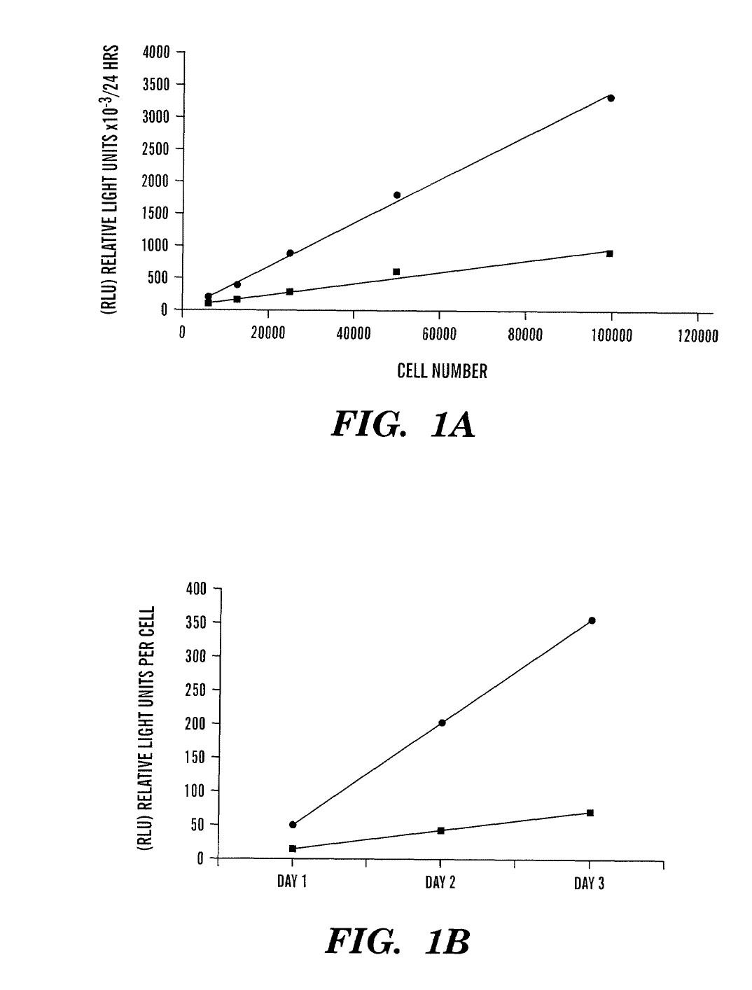

In order to monitor protein secretion from cells, a lentivirus vector was generated in which expression of a Gluc-IRES-cerulean cassette was inserted under control of the CMV promoter. When fibroblasts were infected with this vector at an M.O.I.=50 over 90% of cells began expressing cerulean within 24 hrs. Seventy-two hrs after infection cells were replated, allowed to attach for 24 hrs, then the medium was replaced and levels of Gluc in the medium were assessed over the next 24 hrs. Levels of Gluc activity released into the medium was proportional to cell number (FIG. 1A). To assess the effect of other proteins on the levels of Gluc activity, the levels of Gluc activity released into the medium was tested in cells co-express the gene, DYT1, an early onset torsion dystonia gene that a dominantly inherited movement disorder characterized by sustained, involuntary muscle contractions and abnormal posturing (Fahn, 1988). The levels of Gluc activity in these cells was proportional to cell number (FIG. 1A), although the DYT1 cells having a lower rate of secretion as compared to control cells, 1.3 and 5.7 relative light units (RLU)/hr/25,000 cells, respectively. Luciferase activity in the medium also increased in a linear manner between 24 and 72 hrs after replating, again with DYT1 cells having a lower rate of secretion (FIG. 1B).

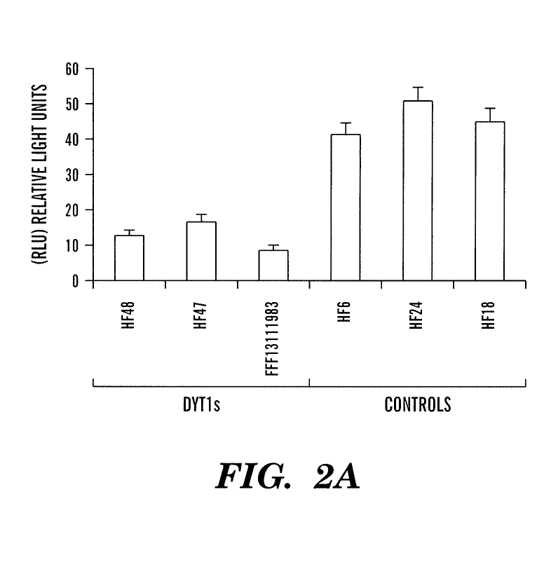

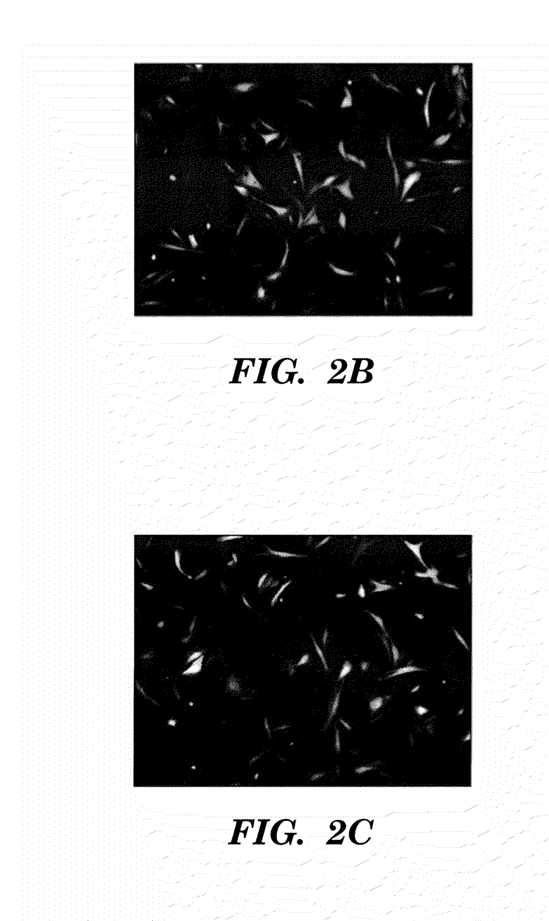

To assess the effect of a mutated protein on the levels of luciferase in the media, luciferase activity was assessed in lenitvirus encoding Gluc infected fibroblasts from normal patients, and DTY1 affected patients which carry a mutation in torsin A. Levels of luciferase activity in the media were evaluated 24 to 48 hrs after replating cells infected with the lentivirus vector encoding Gluc and cerulean using three control lines (genotype=wt/wt torsinA) and three lines from affected DYT1 patients (wt/mt torsinA). Average Gluc activity in the medium of the DYT1 lines was 25% of the average in the control lines (FIG. 2A) with all lines being equally infectable with the lentivirus vector (e.g. FIGS. 2B and C) This difference in average levels of secreted Gluc between the control and DYT1 lines was highly significant (p<0.004). Thus DYT1 fibroblasts, which express both wt and mt torsinA, are compromised in their ability to release active Gluc into the medium as compared to control cells expressing only wt torsinA. Western blot analysis of total levels of torsinA in control and DYT1 lines showed comparable amounts in most control and DYT1 lines (supplementary FIG. 1S) with low levels in one control line, HF18 which did not have a lower level of Gluc secretion (FIG. 2A).

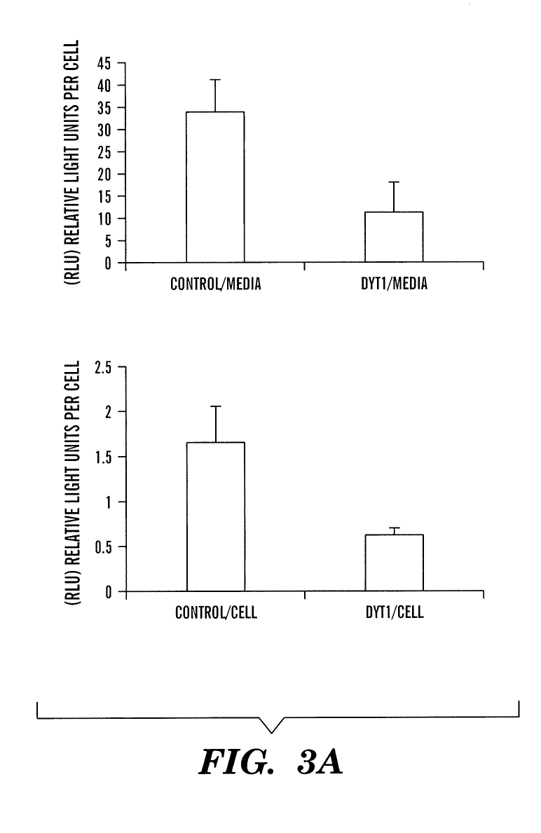

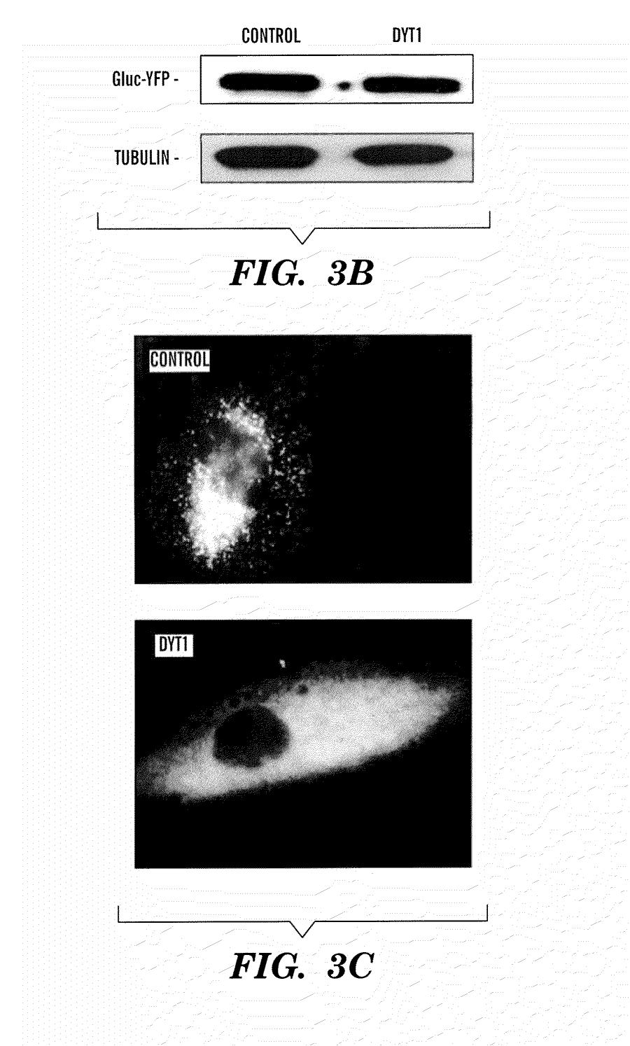

The role of torsinA in processing of Gluc through the secretory pathway was investigated. The pathway could involve entry into the ER, processing within the ER, exit from the ER to the Golgi, or retro-translocation out of the ER into the cytoplasm. To determine the intracellular fate of Gluc in cells, Gluc was fused at its carboxy terminus in-frame to enhanced YFP (a derivative of GFP; Ormo et al., 1996). A decreased level of luciferase activity in the medium (about 30% of control levels) was also found in DYT1 as compared to control fibroblasts expressing this fusion protein (FIG. 3A). This difference in levels of luciferase activity in the medium was accompanied by a parallel and similar decrease in the level of active luciferase in DYT1 cells as compared to control cells, with both cell types having about 95% of activity into the medium (FIG. 3A). Western blot analysis of the Gluc-YFP protein in DYT1 and control cells surprisingly revealed similar levels of the fusion protein in both cell types (FIG. 3B) suggesting that more of the Gluc protein in the DYT1 cells was in an inactive state, as compared to control cells. Therefore Gluc-YFP is not being synthesized at lower levels or preferentially retained within DYT1 cells, but rather is not being processed to an active form as efficiently in DYT1 cells as compared to control cells.

Localization of Gluc-YFP in control and DYT1 cells by immunocytochemistry was also different, with control cells showing a punctuate, reticular pattern of staining characteristic of the ER, while DYT1 cells had a more diffuse distribution, apparently with a cytoplasmic component masking the reticular component (FIG. 3C).

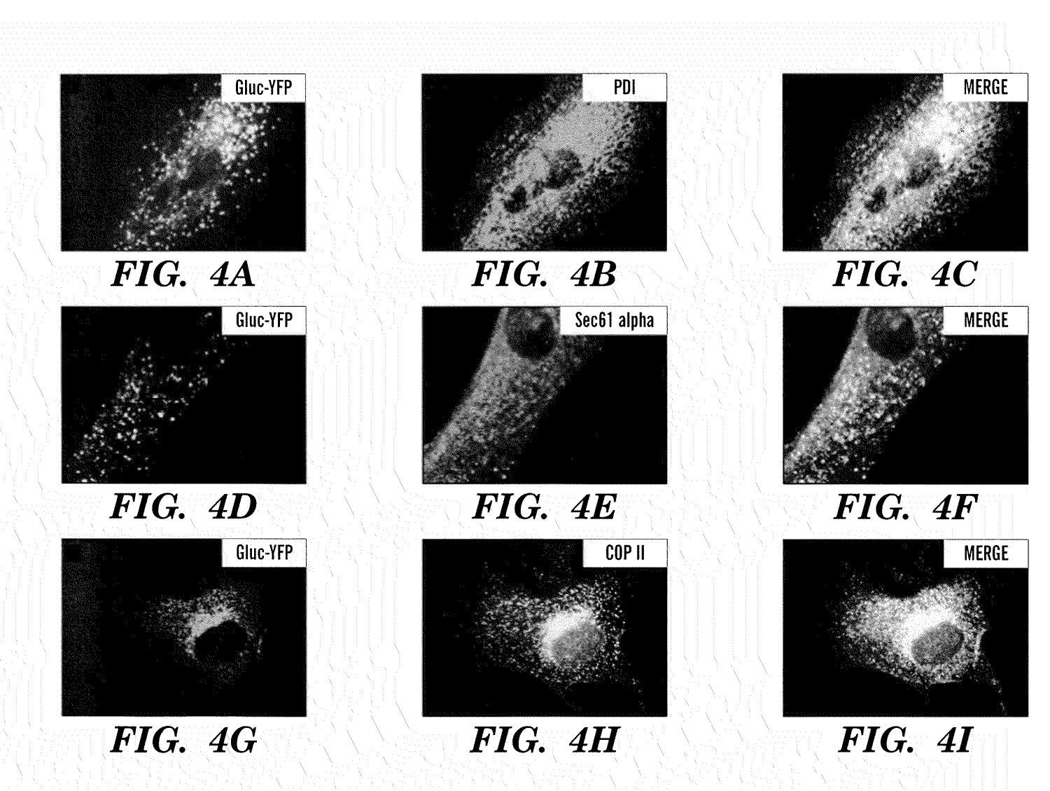

Immunocytochemical studies confirmed that in control cells Gluc-YFP was contained predominantly within the ER by staining for the lumenal ER marker, PDI with the Gluc-YFP staining appearing in a punctuate pattern overlying the ER (FIG. 4A-C). This domain was identified as partially overlapping the translocon domain by staining for ribophorin I (data not shown) and Sec61α (FIG. 4D-F) which are components of the translocon (Pfeffer, 2003). Some co-staining of Gluc puncta was also seen with the ER-Golgi exit marker, COPII (FIG. 4 G-I). These puncta may represent aggregates of Gluc-YFP at regions of increased concentration at the translocon and ER-to-Golgi exit sites promoted by the tendency of YFP to dimerize, like its parent protein, GFP (Snapp et al., 2003).

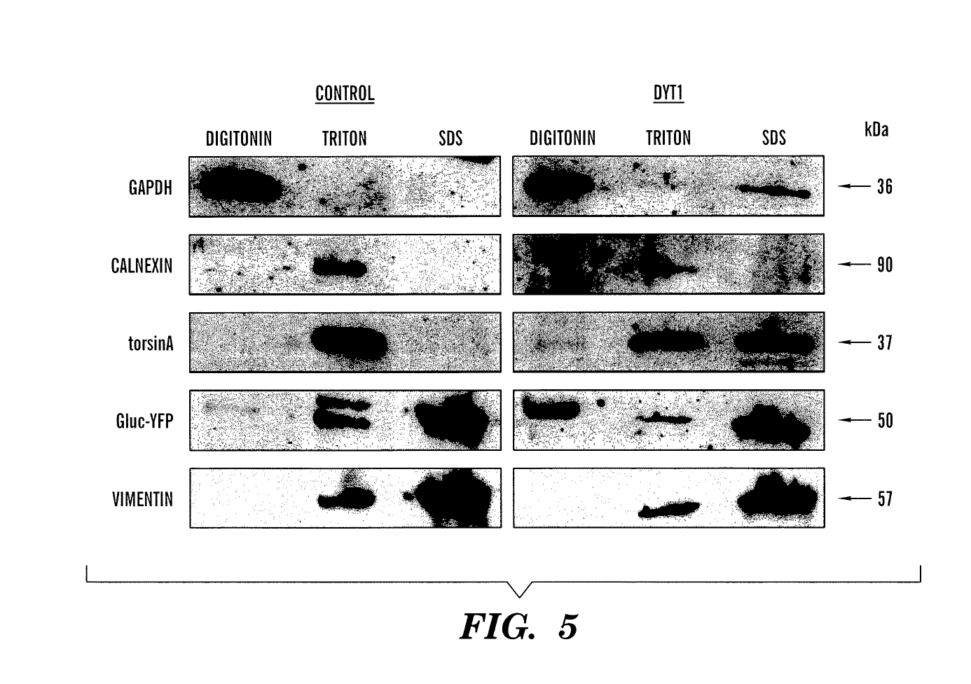

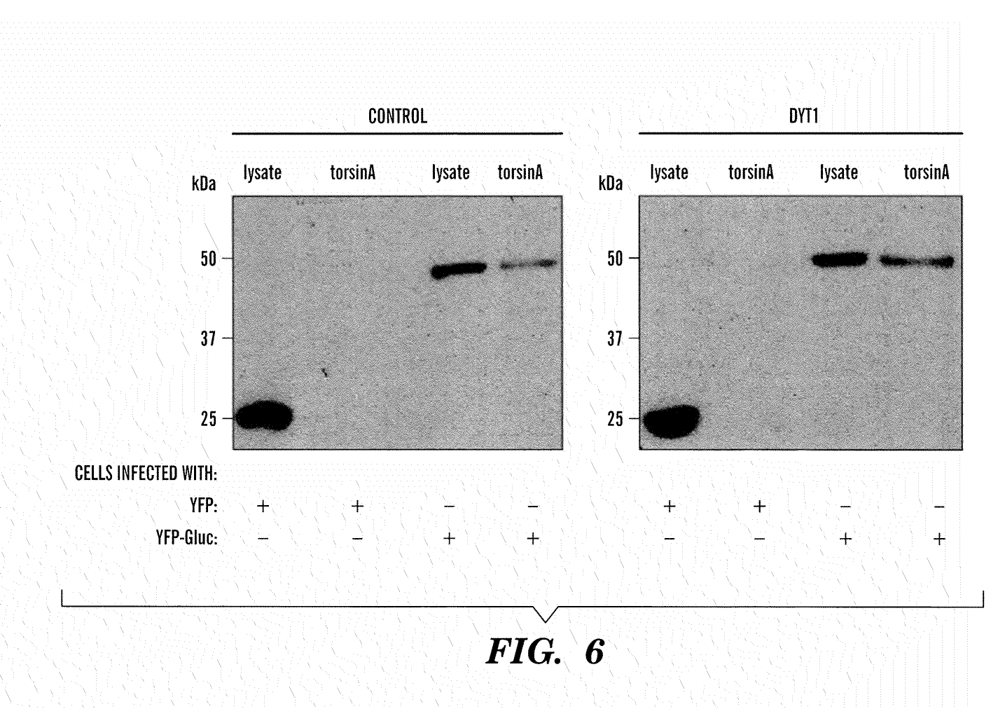

Further evaluation of the distribution of Gluc-YFP in the ER and cytoplasmic fractions was carried out by sequential extraction of control and DYT1 cells with digitonin (cytoplasmic fraction), Triton X-100 (ER fraction) and scraping (residual proteins) using GADPH as a marker for the cytoplasmic fraction, and calnexin and torsinA for the ER fraction. In control cells the marker proteins behaved as predicted (FIG. 5) and Gluc-YFP was found in the ER and residual protein fraction with essentially none in the cytoplasmic fraction. In contrast in DYT1 cells a substantial amount of Gluc-YFP was found in the cytoplasmic fraction as well as in the ER and residual fractions. These findings support the entry into and processing of Gluc-YFP in the ER in control cells, while indicating that this reporter either doesn't enter the ER as efficiently or is retrotranslocated out at a higher rate in DYT1 cells. To determine whether torsinA was associated with Gluc-YFP in cells, co-immune precipitation was carried out using an antibody to torsinA and western blot analysis with antibodies to GFP (FIG. 6). An association between GlucYFP and torsinA was found for both DYT1 and control cells.

To confirm Gluc was processed through the secretory pathway, bioluminescence from the media was measured from cells which had been treated with agents which disrupt the secretory pathway. Bioluminescence was reduced in Gluc expressing cell treated with either Nocoadazone, which disrupts microtubule function, or BrefeldinA which disrupts golgi function compared to untreated cells (FIG. 11). This confirms Gluc is being secreted the secretory pathway.

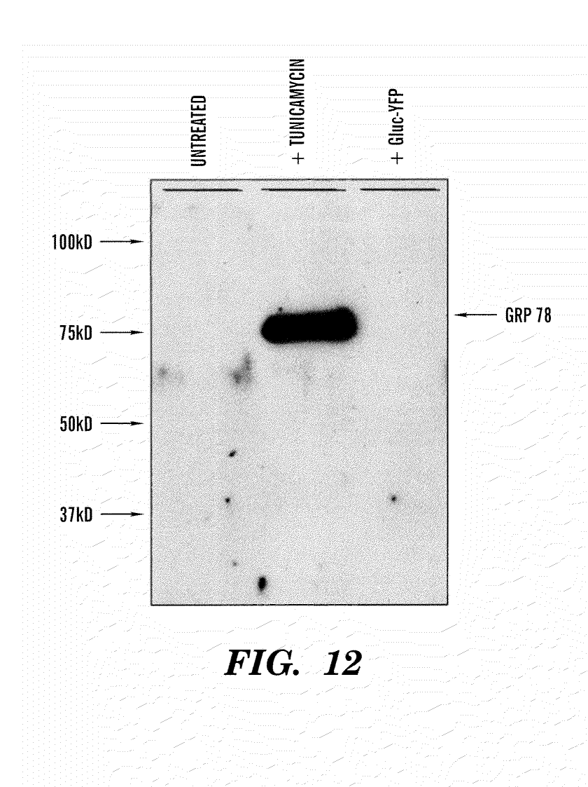

To assess the effect of Gluc expression on cell physiology and to ask if Gluc expression triggers an ER stress response and/or induces the unfolded protein response (UPR), cells were treated with tunicamycin, an agent which upregulates the UPR in the presence or absence of Gluc expression. Expression of GRP78 which is a marker for the BiP protein which is upregulated during the UPR, was detected only in cells treated with tunicamycin, but not in cells expressing the Gluc-YFP fusion protein or tunicamycin untreated cells (FIG. 12). This indicates Gluc expression does not trigger an ER stress response in cells.

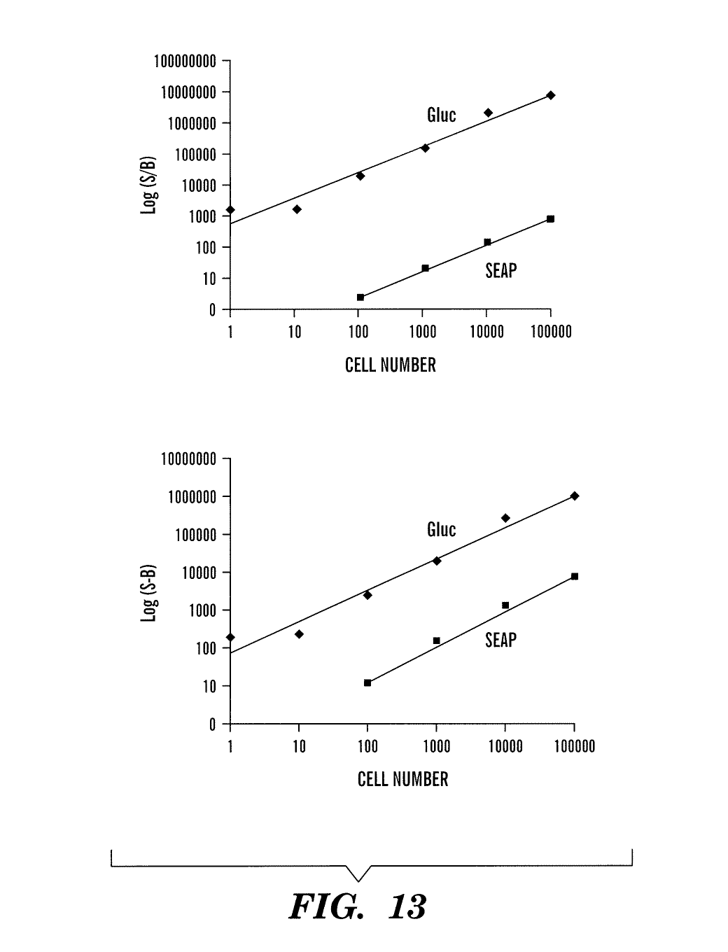

To assess the detection sensitivity of Gluc expression relative to other systems measuring secreted proteins, 3 million 293T human fibroblast cells were transfected with either Gluc or SEAP expressing vectors. 24 hrs after transfection, cells were plated in a dose-dependent manner in 96 well plate. After 24hrs, the conditioned medium was collected from each well and assayed for either Gluc or SEAP activity (FIG. 13). A dose-dependent increase in signal to background ratio (S/B) occurred in both Gluc and SEAP transfected cells, although the signal from Gluc transfected is 1,000-10,000-fold more sensitive than SEAP assay. This indicates secreted Gluc is 1000-10000 fold more sensitive than the SEAP system for measuring secreted proteins.

### Example 2

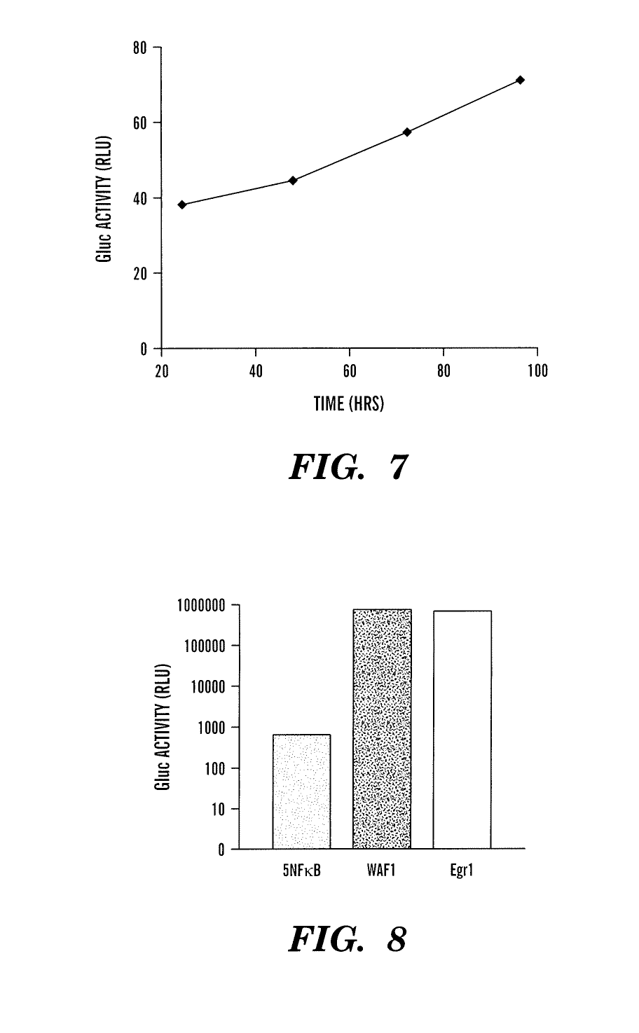

Monitor Promoter Activity. The Gluc assay can also be used to monitor promoter activity with high sensitivity and extended linear range spanning over 7 orders of magnitude. When the Gaussia luciferase cDNA which is codon optimized for mammalian gene expression (hGluc) is expressed under a promoter e.g. CMV, the activity of this promoter can be easily measured overtime by taking an aliquot of the supernatant, adding coelenterazine and measuring the photon counts using a luminometer (FIG. 7). Since the Gaussia luciferase is naturally secreted, the assay is performed on small samples of the conditioned media, with no need to lyse the cells, which makes it much faster and more convenient than assays with other luciferases such as firefly which is used in the SUPERLIGHT™ luciferase reporter gene assay (Bioassays, CA) where cell lysis is required. Since the cells are not disrupted, conditioned media can be sampled repeatedly for time course studies and cells can be used for further studies, such as RNA or protein analysis. This will serve to reduce the variability of transfection efficiency when different wells are used, since only a single well is needed to complete the time course studies. Further, since the Gaussia luciferase is over two thousand fold more sensitive than firefly or Renilla luciferase, it can easily be used to measure promoters over a wide range of activities with no need for signal enhancement for promoters with low activity.

### Example 3

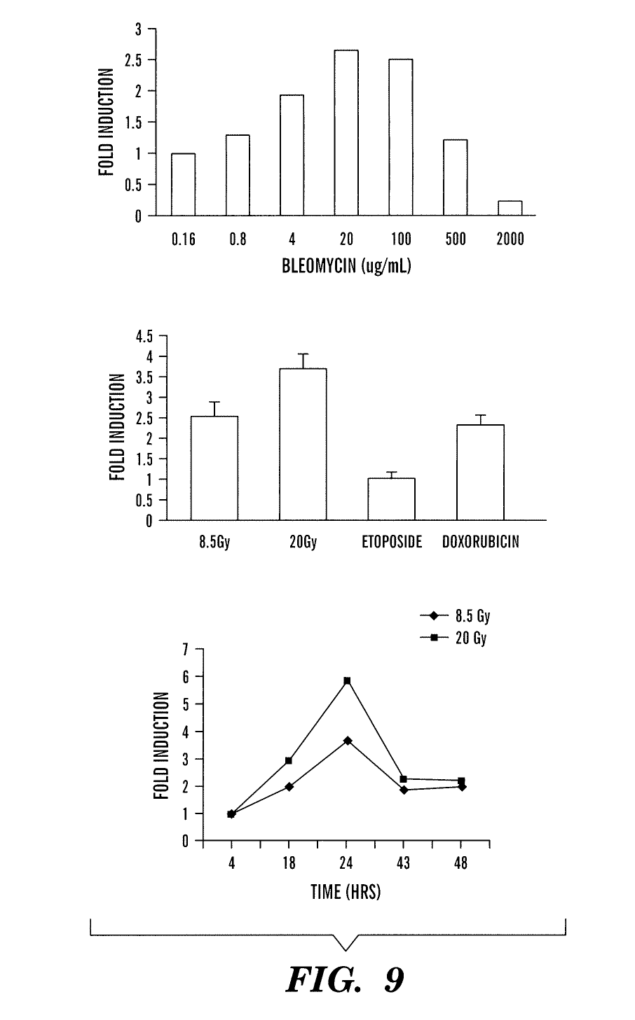

Monitoring signaling pathway and transcriptional activation. The Gluc assay can also be used to monitor signaling pathways and transcriptional activation. The following promoters responsive to different transcription factors were cloned upstream of hGluc cDNA: the wild-type p53-activated fragment 1 (WAF1, 2.4 kb); early growth response factor (Egr-1); and five tandem repeats of the transcriptional factor nuclear factor-κB (5NFκB). Upon transfection with each of these constructs, the activity of each promoter could be monitored by taking an aliquot of the cell medium, adding coelenterazine and measuring photon counts using a luminometer (FIG. 8). Further, the NFκB response to ionizing-radiation and to radiomimetic drugs, such as bleomycin, and chemotherapeutic drugs, such as doxorubicin and etoposide could also be monitored overtime in real-time using hGluc under control of the 5NFκB (FIG. 9).

### Example 4

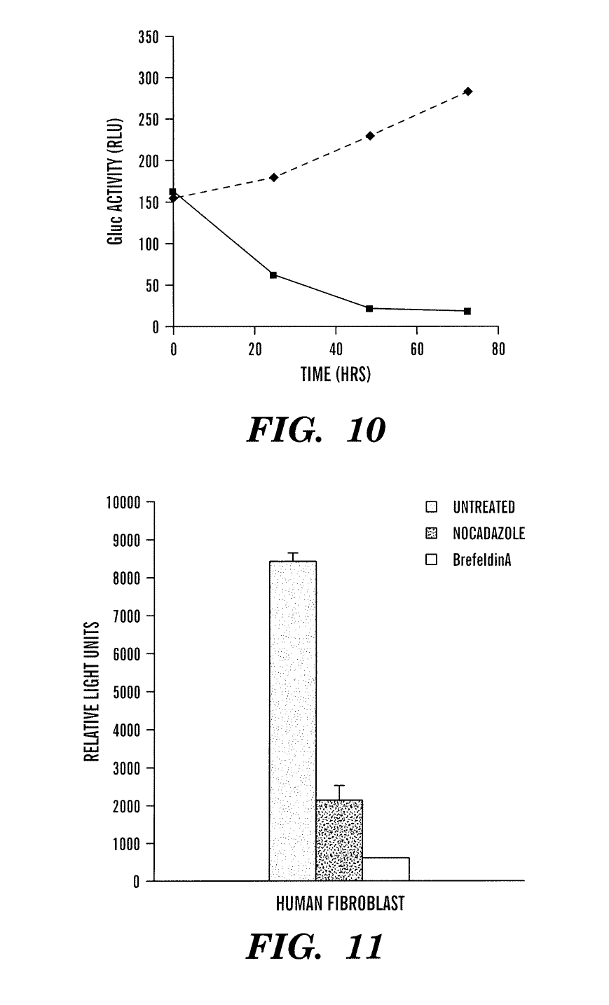

Detection of Cell death. Cell death can also be monitored overtime in the same well by repeated assay of the conditioned media for Gluc activity, since as cells die they stop synthesis and secretion of this reporter (FIG. 10). The cells do not need to be disrupted by the sampling procedure, so the Gluc assay has an advantage over the firefly luciferase assay (Bioassays) in that no cell-lysis is required. Gluc also has an advantage over other apoptosis detection assays, such as CELLQUANTI-MTT™ Cell Viability Assay Kits (Bioassays) as in those cases the reagents need to be added to the cells, which makes it impossible to do a time course in the same well.

### Example 5

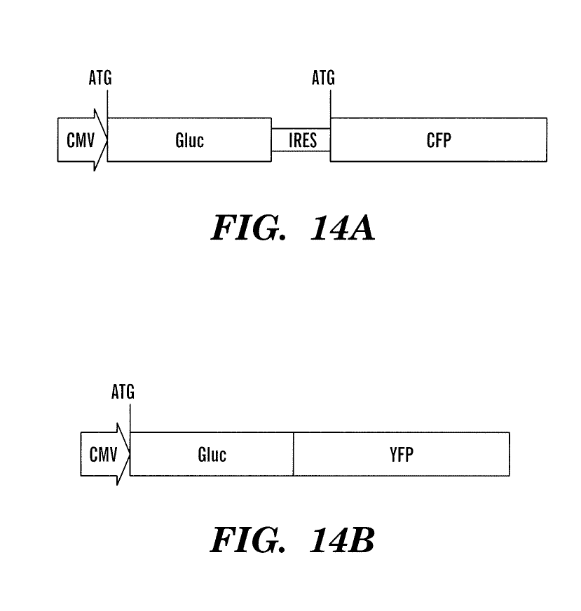

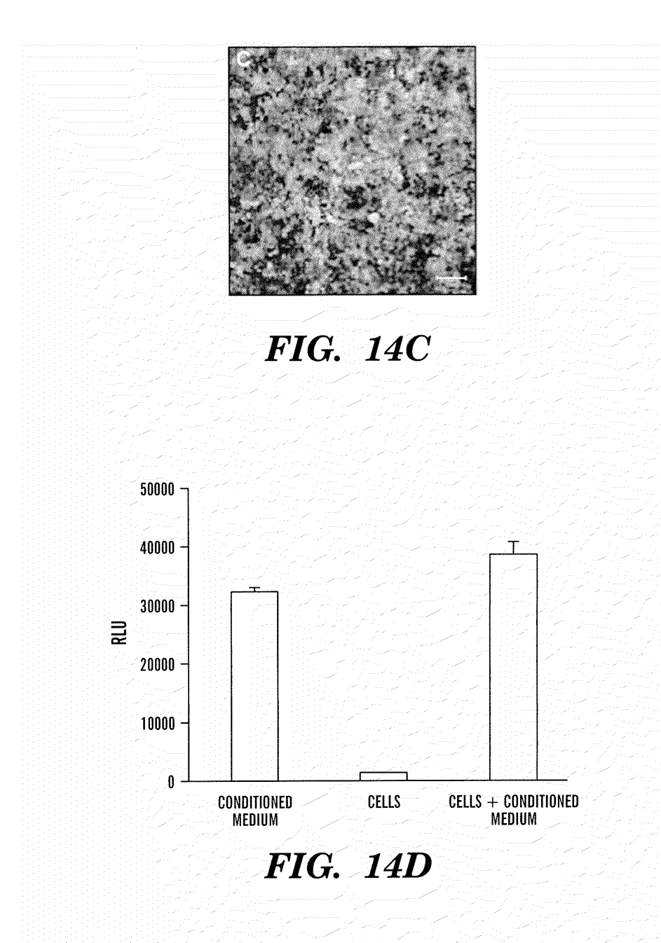

Gluc as a reporter in mammalian cells. A lentivirus vector was generated carrying the expression cassette for humanized Gluc [17] and the blue fluorescent protein, cerulean [18] separated by an internal ribosomal entry site (IRES) (FIG. 14A) and used to deliver Gluc and cerulean to >95% of human fibroblasts (293T cells) in culture by infection (FIG. 14C). In order to assess the level of secreted Gluc with respect to the intracellular level, 293T cells were infected with this lentivirus vector and 24 h later, Gluc activity was assayed in viable cells, the conditioned medium, or both. We observed that >95% of the expressed Gluc is secreted and thus assaying a combination of both intracellular and secreted levels corresponds mostly to the secreted level (FIG. 14D).

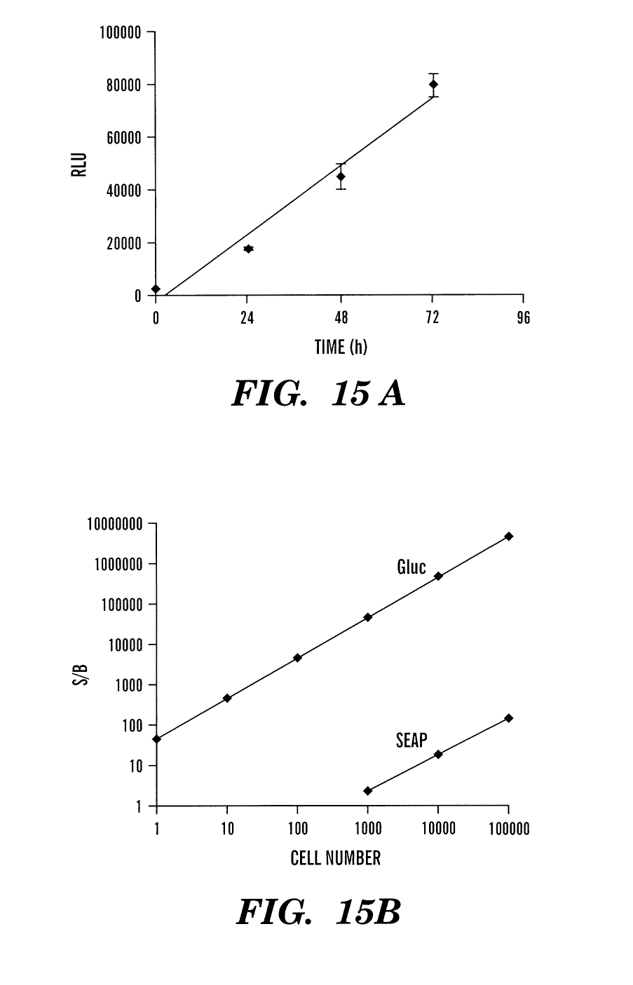

Linearity and sensitivity of Gluc assay. In order to assess the linearity of Gluc with respect to time, 293T cells were infected with the lentivirus vector carrying the expression cassette for Gluc. Forty-eight h post-infection, fresh medium was added and the level of secreted Gluc was assayed overtime by taking an aliquot of the conditioned medium adding coelenterazine, and measuring photon counts using a luminometer. The secreted bioluminescent signal of Gluc from cells into the conditioned medium was linear with respect to time over 72 h postinfection (FIG. 15A).

To compare the linearity of Gluc reporter with respect to cell number and its sensitivity compared to SEAP, a well established assay for monitoring the secretory pathway and ER stress, 293T cells were co-transfected with a plasmid carrying the expression cassette for firefly luciferase (Fluc; for normalization of transfection efficiency) and a plasmid carrying the expression cassette for either Gluc or SEAP. Different numbers of transfected cells ranging from a single cell to one hundred thousand cells were then plated in 96 well plates. Twenty-four h later, luminescent activity of Gluc (measured using coelenterazine) and SEAP (CSPD substrate) was measured in the cell-free conditioned medium, normalizing to the number of transfected cells by Fluc activity (D-luciferin) in cells. Under parallel assay conditions, as low as a single cell could be detected with a signal-to-background ratio (S/B) of 40 with the Gluc assay whereas 20,000 cells were required to get a similar S/B with the SEAP assay (FIG. 15B).

### Example 6

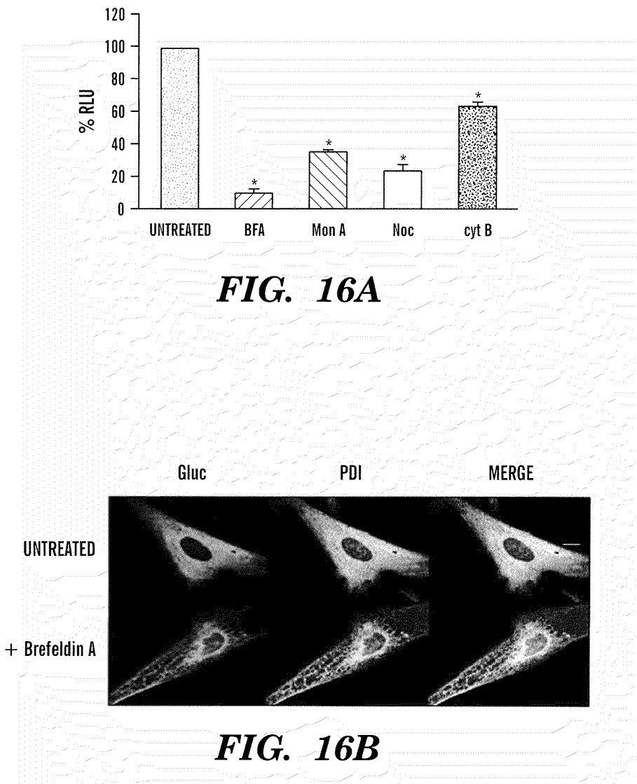

Gaussia luciferase as a reporter for monitoring the secretory pathway. To determine whether Gluc is released via the conventional cell secretory pathway, i.e. rough ER, Golgi and vesicles, we inhibited secretory transport at different steps. 293T cells were treated with the following drugs: brefeldinA (BFA) which inhibits anterograde ER export to the Golgi, but allows retrograde Golgi-ER transport, resulting in a fusion of the ER and the Golgi and blocking of secretion [19]; monensin A which blocks transport within the Golgi [20]; nocodazole which causes microtubule depolymerization leading to blocking of microtubule-dependent translocation of vesicles between the ER and the Golgi [21]; and cytochalasin B which disrupts the actin cytoskeleton and thereby blocks membrane transport along actin filaments [22]. Treating cells with these drugs resulted in significant inhibition of Gluc secretion into the conditioned medium with about 90% decrease with BFA, 65% with monensin A, 75% with nocodazole, and 30% with cytochalasin B at the concentration used over 24 h (FIG. 16A). To investigate the location of Gluc within cells with and without treatment with BFA, HF8 human fibroblast cells expressing Gluc and the same cells treated with BFA were fixed with paraformaldehyde and co-stained with antibodies against Gluc and PDI, an ER marker. In both cases Gluc co-localized with PDI in the ER (FIG. 16B).

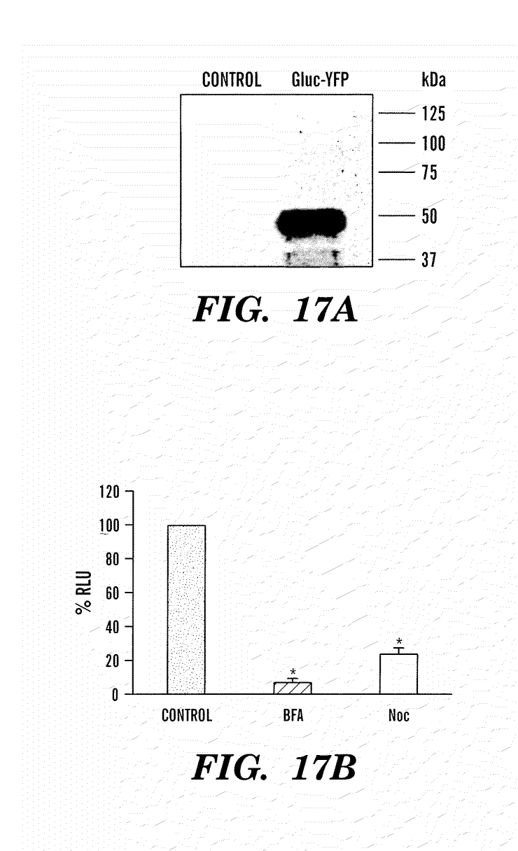

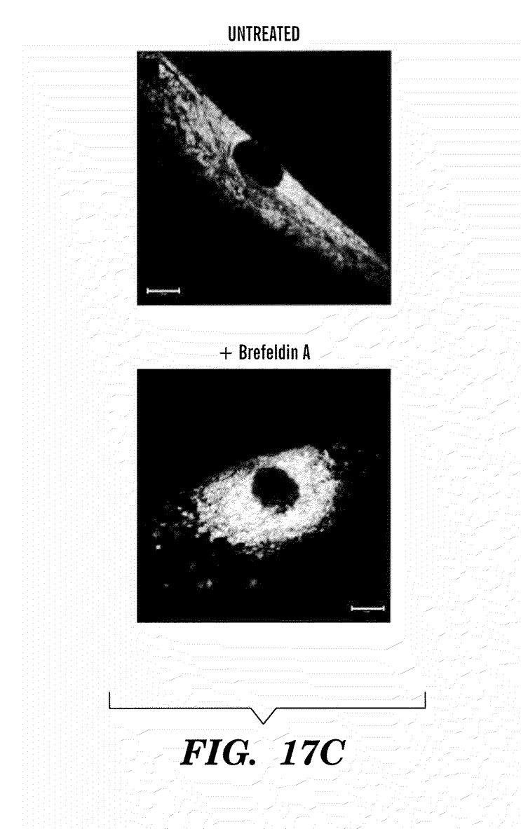

Gluc-YFP fusion to quantitate and visualize secretory pathway in real-time. In order to visualize movement of Gluc fate through the secretory pathway in real-time, we created a fusion protein with Gluc at the N-terminus and YFP at the C-terminus (FIG. 14B). HF8 cells were infected with a lentivirus vector carrying the expression cassette for Gluc-YFP and 48 h later lysates were analyzed by western blotting with anti-Gluc antibody revealing a band at 47 kDa, the predicted size for the fusion protein (FIG. 17A). This same band was also detected with an antibody against GFP (data not shown). Further, when the same cells were treated with BFA and nocodazole, secretion of Gluc-YFP luciferase activity was blocked to the same extent as for Gluc (FIG. 17B) and the fusion protein was also trapped in the ER upon BFA treatment as visualized by real-time confocal microscopy on live cells (FIG. 17C)

### Example 7

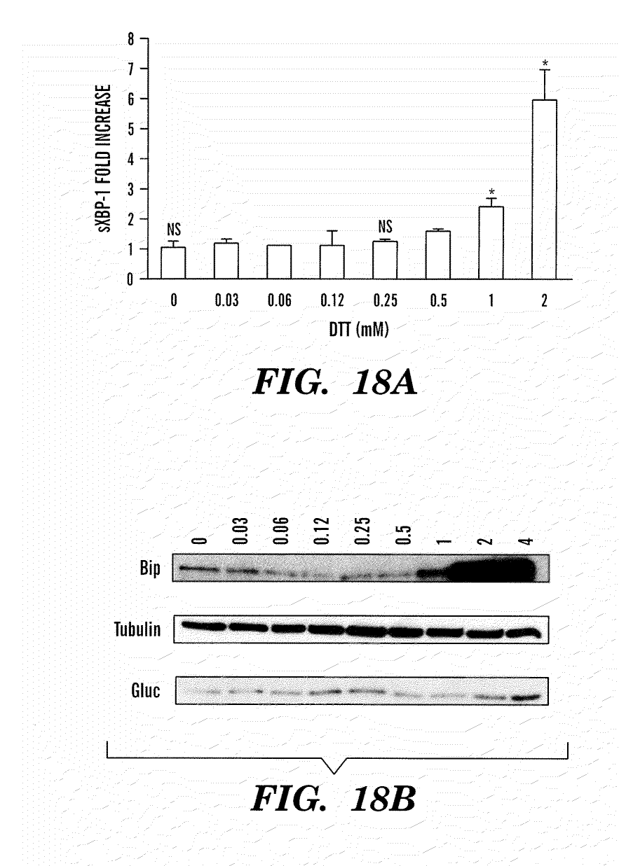

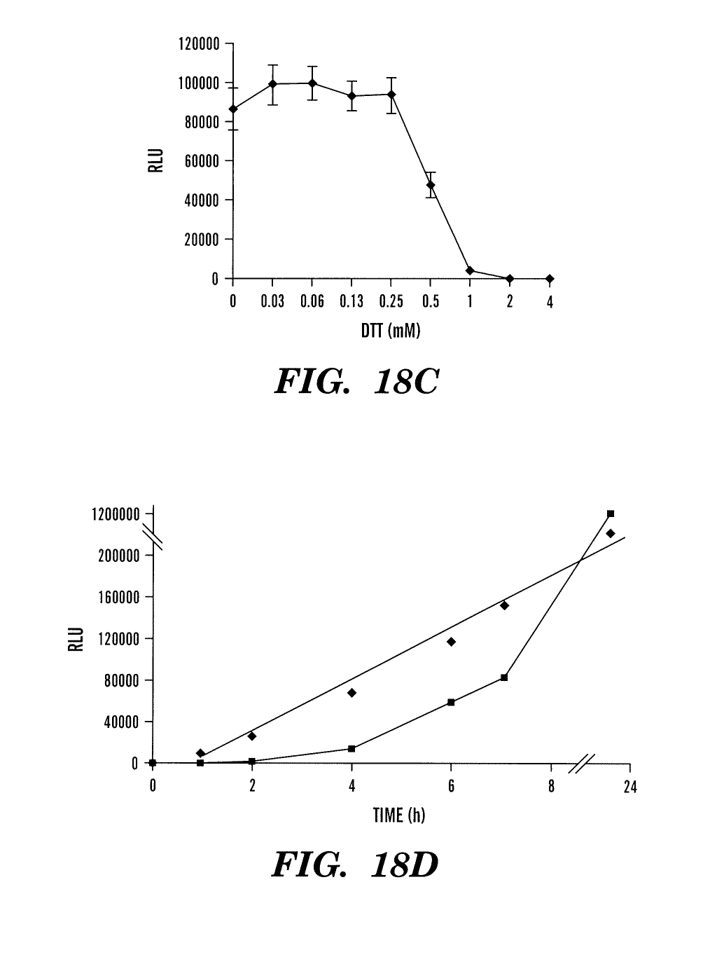

Gaussia luciferase for monitoring of ER stress in real-time. Splicing of the XBP-1 mRNA to sXBP1 and expression of BiP protein are generally used as biomarkers for ER stress [5,6]. Importantly, infection of cells with the lentivirus vector expressing the Gluc reporter did not induce ER stress in and of itself as evaluated by XBP-1 message splicing (FIG. 18A) and Bip levels (FIG. 18B), not did it effect on cell viability as assessed by tetrazolium salt, WST-1 cell proliferation assay (Roche Diagnostics GmbH, Mannheim, Germany) evaluated 48 h after infection of >95% of cells (data not shown). In order to investigate whether changes in Gluc secretion could be used as a marker for ER stress, 293T cells expressing Gluc were treated with different concentration of dithiothreitol (DTT; 0.03-2 mM) which induces ER stress [23]. Then three assays were carried out in parallel: 1) the cellular RNA was subjected to qRT-PCR for sXBP-1 (4 h exposure to DTT); 2) the cell lysates were resolved on SDS-PAGE followed by western blotting for BiP, alpha-tubulin and Gluc (24 h); and 3) the conditioned medium was assayed for Gluc activity (4 h). Increasing the concentration of DTT increased the extent of XBP-1 mRNA splicing (FIG. 18a) and the amount of BiP (FIG. 18b) in a dose dependent manner, with both being inversely correlated with a decrease in secretion of Gluc (FIG. 18c). When tunicamycin (3 μg/ml for 4 h) was used to induce ER stress, similar results were obtained with a 9-fold increase in spliced XBP-1 message and over 90% decrease in Gluc secretion (data not shown). Further, real-time monitoring of Gluc secretion in the conditioned medium upon 1 mM DTT treatment showed a complete inhibition of secretion for the first 3 h which started to recover after 4 h, and returned to normal level after 24 h (FIG. 5d). These findings show that the Gluc reporter is a sensitive marker in elucidating ER stress in mammalian cells in real-time.

The facility of Gluc in reporting on the secretory pathway in mammalian cells by assaying their conditioned medium makes it a highly useful tool in monitoring processing of proteins and detecting ER stress. The Gluc assay as disclosed herein is highly sensitive, quantitative and linearly related with respect to cell number in a range covering over five orders of magnitudes. The Use of Gluc assay as disclosed herein has clear advantages as compared to other systems used to measure the secretory pathway and ER stress, such as the SEAP assay, which is generally used to monitor ER stress [9,16,24] as although the SEAP assay is quantitative, is use has numerous limitations, for example but not limited to (i) it is significantly less sensitive than the Gluc assay, (ii) it is more time consuming to measure levels of activity, and (iii) requires sample dilution and different incubation times at different temperature before processing.

Further, unlike the SEAP assay, the Gluc-YFP embodiment of this reporter system as disclosed herein allows the visualization of the secretory pathway in cells. A well established assay for visualizing ER to Golgi transport is a fusion between VSVG and GFP by switching the temperature from 40° C. at which the fusion is retained in the ER to 32° C. at which the fusion is transported from the ER to the Golgi [10], which allows passage or proteins through the secretory pathway to be arrested by a temperature shift and viewed at the intracellular level, it is limited in its allow quantitative assessment of secretion. In contrast, Gluc-YFP assay as disclosed herein allows visualization and quantitation of proteins through the secretory pathway, which can be assessed drugs that block the secretory pathway.

Recently, another naturally secreted luciferase from the marine copepod Metridia longo has been characterized [25] and used as a marker for protein processing and ER stress. However, Metridia luciferase was shown to be less sensitive than the SEAP assay [16,24] and the SEAP assay is much less sensitive (over 20,000-fold) than the Gluc assay described here. The naturally secreted Gluc reporter as disclosed herein provides a facile, sensitive assay for monitoring the secretory pathway and ER stress and the expression cassette for it can be delivered to cells by either transfection and infection, with the latter expanding the number of cell types that can be tested and allowing for stable expression in progeny cells. Further, the assay as discloser herein is compatible with high throughput drug screening since measurement of secretion only requires the substrate (i.e. coelenterazine) and a plate luminometer which measures its reaction in few seconds, without the need to remove the conditioned medium (since >95% of Gluc is secreted), thus preventing sample contamination, and also allows the measurement to be performed in the same well where cells are plated.

As disclosed herein, the Gluc secretion assay provides a highly sensitive, facile way to monitor processing of proteins through the secretory pathway. Accordingly, the inventors have discovered it is useful to dissect out components of the secretory pathway, for example in elucidating the action of mutant torsinA responsible for early onset torsion dystonia in interfering with protein secretion [26] and in monitoring ER stress, which is implicated in a number of diseases, for example diabetes and neurodegeneration [6]. The inventors have also discovered that this assay is useful to identify drugs which can counteract the effects of mutant proteins using high throughput assays, as well as its use for monitoring effects of drugs and other agents on ER stress in vivo.

### REFERENCES

The references cited herein and throughout the application are incorporated herein by reference.
1. Ellgaard L, Helenius A (2003) Quality control in the endoplasmic reticulum. Nat Rev Mol Cell Biol 4: 181-191.
2. Voeltz G K, Rolls M M, Rapoport T A (2002) Structural organization of the endoplasmic reticulum. EMBO Rep 3: 994-950.
3. Stevens F J, Argon Y (1999) Pathogenic light chains and the B-cell repertoire. Immunol Today 20: 451-457.
4. Tsai B, Ye Y, Rapoport T A (2002) Retro-translocation of proteins from the endoplasmic reticulum into the cytosol. Nat Rev Mol Cell Biol 3: 246-255.
5. Zhang K, Kaufman R J (2006) The unfolded protein response: a stress signaling pathway critical for health and disease. Neurology 66: S102-109.
6. Zhao L, Ackerman S L (2006) Endoplasmic reticulum stress in health and disease. Curr Opin Cell Biol 18: 444-452.
7. Bertrand F, Veissiere D, Hermelin B, Paul A, Capeau J, et al. (1994) Phosphorylation of vimentin is an intermediate step in protein kinase C-mediated glycoconjugate secretion. Am J Physiol 266: C611-621.
8. de Silva A M, Balch W E, Helenius A (1990) Quality control in the endoplasmic reticulum: folding and misfolding of vesicular stomatitis virus G protein in cells and in vitro. J Cell Biol 111: 857-866.
9. Berger J, Hauber J, Hauber R, Geiger R, Cullen B R (1988) Secreted placental alkaline phosphatase: a powerful new quantitative indicator of gene expression in eukaryotic cells. Gene 66: 1-10.
10. Presley J F, Cole N B, Schroer T A, Hirschberg K, Zaal K J, et al. (1997) ER-to-Golgi transport visualized in living cells. Nature 389: 81-85.
11. Lee Y S, Kim H K, Chung S, Kim K S, Dutta A (2005) Depletion of human micro-RNA miR-125b reveals that it is critical for the proliferation of differentiated cells but not for the down-regulation of putative targets during differentiation. J Biol Chem 280: 16635-16641.
12. Hirota M, Kitagaki M, Itagaki H, Aiba S (2006) Quantitative measurement of spliced XBP1 mRNA as an indicator of endoplasmic reticulum stress. J Toxicol Sci 31: 149-156.
13. Li F, Hayashi T, Jin G, Deguchi K, Nagotani S, et al. (2005) The protective effect of dantrolene on ischemic neuronal cell death is associated with reduced expression of endoplasmic reticulum stress markers. Brain Res 28: 59-68.
14. Mao C, Dong D, Little E, Luo S, Lee A S (2004) Transgenic mouse model for monitoring endoplasmic reticulum stress in vivo. Nat Med 10: 1013-1014; author reply 1014.
15. Iwawaki T, Akai R, Kohno K, Miura M (2004) A transgenic mouse model for monitoring endoplasmic reticulum stress. Nat Med 10: 98-102.
16. Hiramatsu N, Kasai A, Hayakawa K, Yao J, Kitamura M (2006) Real-time detection and continuous monitoring of ER stress in vitro and in vivo by ES-TRAP: evidence for systemic, transient ER stress during endotoxemia. Nucleic Acids Res 34: e93.
17. Tannous B A, Kim D E, Fernandez J L, Weissleder R, Breakefield X O (2005) Codon-optimized Gaussia luciferase cDNA for mammalian gene expression in culture and in vivo. Mol Ther 11: 435-443.
18. Rizzo M A, Springer G H, Granada B, Piston D W (2004) An improved cyan fluorescent protein variant useful for FRET. Nat Biotechnol 22: 445-449.
19. Lippincott-Schwartz J, Yuan L C, Bonifacino J S, Klausner R D (1989) Rapid redistribution of Golgi proteins into the ER in cells treated with brefeldin A: evidence for membrane cycling from Golgi to ER. Cell 56: 801-813.
20. Tartakoff A, Vassalli P, Detraz M (1978) Comparative studies of intracellular transport of secretory proteins. J Cell Biol 79: 694-707.
21. Rogalski A A, Singer S J (1984) Associations of elements of the Golgi apparatus with microtubules. J Cell Biol 99: 1092-1100.
22. Forer A, Emmersen J, Behnke O (1972) Cytochalasin B: does it affect actin-like filaments? Science 175: 774-776.
23. Shang J (2005) Quantitative measurement of events in the mammalian unfolded protein response. Methods 35: 390-394.
24. Hiramatsu N, Kasai A, Meng Y, Hayakawa K, Yao J, et al. (2005) Alkaline phosphatase vs luciferase as secreted reporter molecules in vivo. Anal Biochem 339: 249-256.
25. Markova S V, Golz S, Frank L A, Kalthof B, Vysotski E S (2004) Cloning and expression of cDNA for a luciferase from the marine copepod Metridia longa. A novel secreted bioluminescent reporter enzyme. J Biol Chem 279: 3212-3217.
26. Hewett J W, Tannous, B. A., Niland, B. B., Nery, F. C., Zeng, J., Li, Y., Breakefield, X. O. (in press) Mutant torsinA interferes with protein processing through the secretory pathwat in DYT1 dystonia cells. Proc Natl Acad Sci USA.
27. Pear W S, Nolan G P, Scott M L, Baltimore D (1993) Production of high-titer helper-free retroviruses by transient transfection. Proc Natl Acad Sci USA 90: 8392-8396.
28. Breakefield X O, Braverman M, Riker D K, Giller E L, Jr. (1981) Catechol-O-methyltransferase activity in cultured human skin fibroblasts from controls and patients with dystonia musculorum deformans. J Neurosci Res 6: 349-360.
29. Sena-Esteves M, Tebbets J C, Steffens S, Crombleholme T, Flake A W (2004) Optimized large-scale production of high titer lentivirus vector pseudotypes. J Virol Methods 122: 131-139.
30. Osbourne et al.(2005) Protein translocation by the Sec61/SecY channel. Annu. Rev. Cell Dev. Biol. 21: 529-550.
31. Presley, J. F., Cole, N. B., Schroer, T. A., Hirschberg, K., Zaal, K. J. and Lippincott-Schwartz, J.: ER-to-Golgi transport visualized in living cells. Nature 389 (1997) 81-85.
32. Tannous B A, Kim D E, Fermandez J L, Weissleder R, Breakefield X O (2005) Codon optimized Gaussia luciferase cDNA for mammalian gene expression in culture and in vivo. Mol Ther 11:435-43.
33. Tsai et al., (2002) Retro-translocation of proteins from the endoplasmic reticulum to the cytosol. Nat. Rev. Mol. Cell Biol. 3: 246-255.
34. Yang, T. T., Sinai, P., Kitts, P. A. and Kain, S. R.: Quantification of gene expression with a secreted alkaline phosphatase reporter system. Biotechniques 23 (1997) 1110-1114
35. Asanuma, K., Carbon-Correll, M. & Eidelberg, D. (2005) J Med Invest 52, 272-279.
36. Augood, S. J., Martin, D. M., Ozelius, L. J., Breakefield, X. O., Penney, J. B. J. & Standaert, D. G. (1999) Ann Neurol 46, 761-769.
37. Basham, S. E. & Rose, L. S. (2001) Development 128, 4645-4656.
38. Bermano, G., Shepherd, R. K., Zehner, Z. E. & Hesketh, J. E. (2001) FEBS Lett 497, 77-81.
39. Blood, A. J., Flaherty, A. W., Choi, J. K., Hochberg, F. H., Greve, D. N., Bonmassar, G., Rosen, B. R. & Jenkins, B. G. (2004) Ann Neurol 55, 744-748.
40. Bragg, D. C., Camp, S. M., Kaufman, C. A., Wilbur, J. D., Boston, H., Schuback, D. E., Hanson, R. I., Sena-Esteves, M. & Breakefield, X. O. (2004) Neurosci 125, 651-661.
41. Breakefield, X. O. & Standaert, D. G. New Encycl Neurosci, in press.
42. Breakefield, X. O., Braverman, M., Riker, D. K. & Giller, E. L. J. (1981) J Neurosci Res 6, 349-360.
43. Caldwell, G. A., Cao, S., Sexton, E. G., Gelwix, C. C., Bevel, J. P. & Caldwell, K. A. (2003) Hum Mol Genet 12, 307-319.
44. Cao, S., Gelwix, C. C., Caldwell, K. A. & Caldwell, G. A. (2005) J Neurosci 25, 3801-3812.
45. Chabanon, H., Mickleburgh, I. & Hesketh, J. (2005) Brief Funct Genomic Proteomic 3, 240-256.
46. Clark, J. E., Brennan, A., Ramesh, T. M. & Heywood, J. A. (2002) Front Biosci 7, c83-96.
47. Cohen, R. S. (2005) Biol Cell 97, 5-18.
48. D'Angelo, M. A. & Hetzer, M. W. (2006) Cell Mol Life Sci 63, 316-332.
49. Dang, M. T., Yokoi, F., McNaught, K. S., Jengelley, T. A., Jackson, T., Li, J. & Li, Y. (2005) Exp Neurol 196, 452-463.
50. Djabali, K., Piron, G., de Nechaud, B. & Portier, M. M. (1999) Exp Cell Res 253, 649-662.
51. Ellgaard, L. & Helenius, A. (2003) Nat Rev Mol Cell Biol 4, 181-191.
52. Fahn, S. (1988) Adv Neurol 50, 1-8.
53. Fahn, S., Burke, R. & Stem, Y. (1990) Prog Brain Res 84, 389-397.
54. Ferrari-Toninelli, G., Paccioretti, S., Francisconi, S., Uberti, D. & Memo, M. (2004) Brain Res 1012, 75-81.
55. Goodchild, R. E. & Dauer, W. T. (2004) Proc Natl Acad Sci USA 101, 847-852.
56. Goodchild, R. E. & Dauer, W. T. (2005) J Cell Biol 168, 855-862.
57. Goodchild, R. E., Kim, C. E. & Dauer, W. T. (2005) Neuron 48, 923-932.
58. Hanson, P. I. & Whitehouse, S. W. (2005) Nat Rev Mol Cell Biol 6, 519-529.
59. Hedreen, J. C., Zweig, R. M., DeLong, M. R., Whitehouse, P. J. & Price, D. L. (1988) Adv Neurol 50, 123-132.
60. Heemskerk, J. (2005) Retina 25, S56-S57.
61. Hewett, J., Gonzalez-Agosti, C., Slater, D., Ziefer, P., Li, S., Bergeron, D., Jacoby, D. J., Ozelius, L. J., Ramesh, V. & Breakefield, X. O. (2000) Hum Mol Genet 9, 1403-1413.
62. Hewett, J., Ziefer, P., Bergeron, D., Naismith, T., Boston, H., Slater, D., Wilbur, J., Schuback, D., Kamm, C., Smith, N., Camp, S., Ozelius, L. J., Ramesh, V., Hanson, P. I. & Breakefield, X. O. (2003) J Neurosci Res 72, 158-168.
63. Hewett, J. W., Kamm, C., Boston, H., Beauchamp, R., Naismith, T., Ozelius, L., Hanson, P. I., Breakefield, X. O. & Ramesh, V. (2004) J Neurochem 89, 1186-1194.
64. Hewett, J. W., Zeng, J., Niland, B. P., Bragg, D. C. & Breakefield, X. O. (2006) Neurobiol Dis 22, 98-111.
65. Kabakci, K., Hedrich, K., Leung, J. C., Mitterer, M., Vieregge, P., Lencer, R., Hagenah, J., Garrels, J., Witt, K., Klostermann, F., Svetel, M., Friedman, J., Kostic, V., Bressman, S., Breakefield, X. O., Ozelius, L. J., Pramstaller, P. P. & Klein, C. (2004) Neurol 62, 395-400.
66. Kamm, C., Boston, H., Hewett, J., Wilbur, J., Corey, D. P., Hanson, P. I., Ramesh, V. & Breakefield, X. O. (2004) J Biol Chem 279, 19882-19892.
67. Kislauskis, E. H., Zhu, X. & Singer, R. H. (1997) J Cell Biol 136, 1263-1270.
68. Kock, N., Allchorne, A. J., Sena-Esteves, M., Woolf, C. J. & Breakefield, X. O. (2006) Neurosci Lett 395, 201-205.
69. Kopito, R. R. & Sitia, R. (2000) EMBO Rep 1, 225-231.
70. Kuner, R., Teismann, P., Trutzel, A., Naim, J., Richter, A., Schmidt, N., von Ahsen, O., Bach, A., Ferger, B. & Schneider, A. (2003) Neurosci Lett. 350, 153-156.
71. Kustedjo, K., Bracey, M. H. & Cravatt, B. F. (2000) J Biol Chem 275, 27933-27939. Levine, C. G., Mitra, D., Sharma, A., Smith, C. L. and Hegde, R. S. (2005) Mol Cell Biol 16, 279-291.
72. Liu, Z., Zolkiewska, A. & Zolkiewska, M. (2003) Biochem J 374, 117-122.
73. McLean, P. J., Kawamata, H., Shariff, S., Hewett, J., Sharma, N., Ueda, K., Breakefield, X. O. & Hyman, B. T. (2002) J Neurochem 83, 846-854.
74. McNaught, K. S., Kapustin, A., Jackson, T., Jengelley, T. A., Jnobaptiste, R., Shashidharan, P., Perl, D. P., Pasik, P. & Olanow, C. W. (2004) Ann Neurol 56, 540-547.
75. Misbahuddin, A., Placzek, M. R., Taanman, J. W., Gschmeissner, S., Schiavo, G., Cooper, J. M. & Warner, T. T. (2005) Mov Disord 20, 432-440.
76. Molinari, M. & Sitia, R. (2006) CTMIA 300, 1-15
77. Naismith, T. V., Heuser, J. E., Breakefield, X. O. & Hanson, P. I. (2004) Proc Natl Acad Sci USA 101, 7612-7617.
78. Neuwald, A. F., Aravind, L., Spouge, J. L. & Koonin, E. V. (1999) Genomic Res 9, 27-43.
79. O'Farrell, C., Lockhart, P. J., Lincoln, S., De Lucia, M., Singleton, A. B., Dickson, D. W. & Cookson, M. R. (2004) Brain Res Mol Brain Res 127, 1-9.
80. Ogura, T. & Wilkinson, A. J. (2001) Genes Cells 6, 575-597.
81. Ormo, M., Cubitt, A. B., Kallio, K., Gross, L. A., Tsien, R. Y. & Remington, S. J. (1996) Science 273, 1392-1395.
82. Osborne, A. R., Rapoport, T. A. & van den Berg, B. (2005) Annu Rev Cell Dev Biol 21, 529-550.
83. Ozelius, L. J., Hewett, J. W., Page, C. E., Bressman, S. B., Kramer, P. L., Shalish, C., de Leon, D., Brin, M. F., Raymond, D., Corey, D. P., Fahn, S., Risch, N. J., Buckler, A. J., Gusella, J. F. & Breakefield, X. O. (1997) Nat Genet 9, 27-43.
84. Palmer, K. J., Watson, P. & Stephens, D. J. (2005) Biochem Soc Symp 72, 1-13.
85. Pfeffer, S. (2003) Cell 112, 507-517.
86. Presley, J. F., Cole, N. B., Schroer, T. A., Hirschberg, K., Zaal, K. J. & Lippincott-Schwartz, J. (1997) Nature 389, 81-85.
87. Quartarone, A., Siebner, H. R. & Rothwell, J. C. (2006) Trends Neurosci 29, 192-199.
88. Rizzo, M. A., Springer, G. H., Granada, B. & Piston, D. W. (2004) Nat Biotechnol 22, 445-449.
89. Rostasy, K., Augood, S. J., Hewett, J. W., Leung, J. C., Sasaki, H., Ozelius, L. J., Ramesh, V., Standaert, D. G., Breakefield, X. O. & Hedreen, J. C. (2003) Neurobiol Dis 12, 11-24.
90. Sarria, A. J., Lieber, J. G., Nordeen, S. K. & Evans, R. M. (1994) J Cell Sci 107, 1593-1607.
91. Sena-Esteves, M., Tebbets, J. C., Steffens, S., Crombleholme, T. & Flake, A. W. (2004) J Virol Methods 122, 131-139.
92. Sharma, N., Hewett, J., Ozelius, L. J., Ramesh, V., McLean, P. J., Breakefield, X. O. & Hyman, B. T. (2001) Am J Pathol 159, 339-344.
93. Shashidharan, P., Paris, N., Sandu, D., Karthikeyan, L., McNaught, K. S., Walker, R. H. & Olanow, C. W. (2004) J Neurochem 88, 1019-1025.
94. Snapp, E. L., Hegde, R. S., Francolini, M., Lombardo, F., Colombo, S., Pedrazzini, E., Borgese, N. & Lippincott-Schwartz, J. (2003) J Cell Biol 163, 257-269.
95. Stevens, F. J. & Argon, Y. (1999) Immunol Today 20, 451-457.
96. Tanaka, M., Machida, Y. & Nukina, N. (2005) J Mol Med 83, 343-352.
97. Tannous, B. A., Kim, D. E., Fernandez, J. L., Weissleder, R. & Breakefield, X. O. (2005) Mol Ther 11, 435-443.
98. Toda, H., Hamani, C. & Lozano, A. (2004) Neurosurg Focus 17, E2.
99. Torres, G. E., Sweeney, A. L., Beaulieu, J. M., Shashidharan, P. & Caron, M. G. (2004) Proc Natl Acad Sci USA 101, 15650-15655.
100. Tsai, B., Ye, Y. & Rapoport, T. A. (2002) Nat Rev Mol Cell Biol 3, 246-255.
101. Vale, R. D. (2000) J Cell Biol 150, F13-19.
102. Vidailhet, M. & Pollack, P. (2005) Ann Neurol 57, 613-614.
103. Voeltz, G. K., Rolls, M. M. & Rapoport, T. A. (2002) EMBO Rep 3, 994-950.
104. Whiteheart, S. W., Rossnagel, K., Buhrow, S. A., Brunner, M., Jaenicke, R. & Rothman, J. E. (1994) J Cell Biol 126, 945-954.
105. Wilhelmsen, K., Litjens, S. H., Kuikman, I., Tshimbalanga, N., Janssen, H., van den Bout, I., Raymond, K. & Sonnenberg, A. (2005) J Cell Biol 171, 799-810.
106. Yang, T. T., Sinai, P., Kitts, P. A. & Kain, S. R. (1997) Biotechniques 23, 1110-1114.
107. Zhang, K. & Kaufman, R. J. (2006) Neurology 66, S102-109.

1. A nucleic acid construct comprising:
a nucleic acid sequence encoding a secreted luciferase-fluorescent protein conjugate;
a nucleic acid sequence encoding at least one regulatory sequence;
wherein the nucleic acid encoding a secreted luciferase-fluorescent protein conjugate is operatively linked to at least one of regulatory sequence.

2. The nucleic acid construct of claim 1 further comprising a nucleic acid sequence encoding at least one multiple cloning site for the introduction of target nucleic acid sequences.

3. The nucleic acid construct of claim 1 further comprising a nucleic acid sequence encoding at least one target nucleic acid sequence.

4. The nucleic acid construct in claim 1 further comprising a nucleic acid sequence encoding at least one internal ribosome entry site (IRES).

5. The nucleic acid construct in claim 1 further comprising at least one nucleic acid sequence encoding a marker gene for identification of cells containing the nucleic acid construct.

6. (canceled)

7. The nucleic acid construct in claim 1, wherein the secreted luciferase-fluorescent protein conjugate is a Gaussia luciferase-fluorescent protein conjugate.

8. The nucleic acid construct of claim 7, wherein a Gaussia luciferase-fluorescent protein conjugate comprises Gaussia luciferase (GLuc) and has the nucleic acid sequence as set forth in SEQ ID No.1.

9. (canceled)

10. The nucleic acid construct of claim 7, wherein the nucleic acid sequence for Gaussia luciferase (GLuc) is humanized Gaussia luciferase (hGLuc) and has the nucleic acid sequence as set forth in SEQ ID No.3.

11.-16. (canceled)

17. A method of analyzing a cell comprising the steps of:
a. introducing the nucleic acid construct of claim 1 into a cell;
b. expressing the secreted luciferase-fluorescent protein conjugate;
c. measuring a fluorescence signal from the secreted luciferase-fluorescent protein conjugate;
and/or
d. measuring the bioluminescence signal from the secreted luciferase-fluorescent protein conjugate.

18. A method of measuring ER-stress in a cell comprising the steps of:
a. introducing the nucleic acid construct of claim 1 into a cell;
b. expressing the secreted luciferase-fluorescent protein conjugate;
c. measuring the level of fluorescence signal from the secreted luciferase-fluorescent protein conjugate; and/or
d. measuring the level of bioluminescence signal from the secreted luciferase-fluorescent protein conjugate,
wherein the level of fluorescence signal from the secreted luciferase-fluorescent protein conjugate and/or level of bioluminescence signal from the secreted luciferase-fluorescent protein conjugate is a measure of ER stress in the cell.

19.-26. (canceled)

27. The method of claim 17, wherein:
a. the nucleic acid construct comprises a regulatory sequence operatively connected to a coding portion encoding the secreted luciferase-fluorescent protein conjugate; and
b. the method further comprising the step of exposing the regulatory sequence to at least one environmental stimulus, whereby fluorescent signal and/or luciferase signal from the secreted luciferase-fluorescent protein conjugate measures an effect of the environmental stimulus on the activity of the regulatory sequence.

28. (canceled)

29. The method of claim 27, wherein a regulatory sequence is selected from a group comprising; promoters; enhancers; 5′ untranslated regions (5′ UTR); 3′ untranslated regions (3′UTRs); and repressor sequences.

30.-42. (canceled)

43. The method of claim 17, wherein the method is used in the study the effects of modification of a regulatory sequence on the activity of a regulatory sequence in the cell.

44. The method of claim 27, wherein the method is used in the study the effects of environmental stimuli on a regulatory sequence and/or promoter activity in the cell.

45. The method of claim 17, wherein the method is used in the study the effects of an intrinsic environmental stimuli on secretory pathways in the cell, wherein any change in the secretory pathway is detected by the fluorescent signal and/or cellular localization of the secreted luciferase-fluorescent protein conjugate or Gaussia luciferase-fluorescent protein conjugate and/or any change in the luciferase signal.

46. The method of claim 27, wherein the method is used in the study the effects of environmental stimuli on secretory pathways in the cell, wherein any change in the secretory pathway is detected by the fluorescent signal and/or cellular localization of the Gaussia luciferase-fluorescent protein conjugate and/or any change in the luciferase signal.

47. The method of claim 17, wherein the method is used in the study the effects of environmental stimuli transcriptional activity in the cell.

48. The method of claim 17, wherein the method is used in the study the effects of environmental stimuli on signaling pathways in the cell.

49. The method of claim 17, wherein the method is used in the study the effects of environmental stimuli on cell viability.

50.-54. (canceled)


SEQUENCE LISTING (13 sequences)

SEQ ID NO: 1 - DNA, 758 bp, Gaussia princeps
ggtactcaaagtatcttctggcagggaaaatgggagtgaaagttctttttgcccttatttgtattgctgtggccgaggccaaaccaactgaaaacaatgaagatttcaacattgtagctgtagctagcaactttgctacaacggatctcgatgctgaccgtggtaaattgcccggaaaaaaattaccacttgaggtactcaaagaaatggaagccaatgctaggaaagctggctgcactaggggatgtctgatatgcctgtcacacatcaagtgtacacccaaaatgaagaagtttatcccaggaagatgccacacctatgaaggagacaaagaaagtgcacagggaggaataggagaggctattgttgacattcctgaaattcctgggtttaaggatttggaacccatggaacaattcattgcacaagttgacctatgtgtagactgcacaactggatgcctcaaaggtcttgccaatgtgcaatgttctgatttactcaagaaatggctgccacaaagatgtgcaacttttgctagcaaaattcaaggccaagtggacaaaataaagggtgccggtggtgattaatcctaatagaatactgcataactggatgatgatatactagcttattgctcataaaatggccattttttgtaacaaatcgagtctatgtaattcaaaatacctaattaattgttaatacatatgtaattcctataaatataatttatgcaatccaaaaaaaaaaaaaaaaaa

SEQ ID NO: 2 - protein, 184 aa, Gaussia princeps
MGVKVLFALICIAVAEAKPTENNEDFNIVAVASNFATTDLDADRGKLPGKKLPLEVLKEMANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 3 - DNA, 558 bp, Gaussia princeps
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactag

SEQ ID NO: 4 - protein, 185 aa, Gaussia princeps
MGVKVLFALICIAVAEAKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 5 - DNA, 1281 bp, Artificial Sequence
  Description of Artificial Sequence Synthetic construct
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgacggtctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 6 - DNA, 30 bp, Artificial Sequence
  Description of Artificial Sequence Synthetic primer
ctcgcggaattcaaaatgaaaccaactgaa

SEQ ID NO: 7 - DNA, 33 bp, Artificial Sequence
  Description of Artificial Sequence Synthetic primer
agctgcctcgaggcattaatcaccaccggcacc

SEQ ID NO: 8 - DNA, 33 bp, Artificial Sequence
  Description of Artificial Sequence Synthetic primer
ctcgcggaattcaaaatgggagtcaaagttctg

SEQ ID NO: 9 - DNA, 33 bp, Artificial Sequence
  Description of Artificial Sequence Synthetic primer
agctgcctcgaggcattactagtcaccaccggc

SEQ ID NO: 10 - DNA, 22 bp, Artificial Sequence
  Description of Artificial Sequence Synthetic primer
ggtctgctgagtccgcagcagg

SEQ ID NO: 11 - DNA, 19 bp, Artificial Sequence
  Description of Artificial Sequence Synthetic primer
gggcttggtatatatgtgg

SEQ ID NO: 12 - DNA, 24 bp, Artificial Sequence
  Description of Artificial Sequence Synthetic primer
tggaaagctgtggcgtgatggccg

SEQ ID NO: 13 - DNA, 26 bp, Artificial Sequence
  Description of Artificial Sequence Synthetic primer
cacccagaagactgtggatggcccct